(19) United States
(12) Patent Application Publication
(10) Pub. No.: US 2022/0033410 A1
(43) Pub. Date: Feb. 3, 2022

(54) SYNTHESIS OF NANOTUBULAR MOLECULES

(71) Applicant: University of Pittsburgh - Of the Commonwealth System of Higher Education, Pittsburgh, PA (US)

(72) Inventors: Raúl Hernández Sánchez, Pittsburgh, PA (US); Saber Mirzaei, Pittsburgh, PA (US); Edison Arley Castro Portillo, Pittsburgh, PA (US)

(21) Appl. No.: 17/386,100

(22) Filed: Jul. 27, 2021

Related U.S. Application Data
(60) Provisional application No. 63/057,506, filed on Jul. 28, 2020.

(51) Int. Cl. C07D 491/22; C07D 403/14; C07D 495/22; C07D 493/22

(52) U.S. Cl. CPC C07D 491/22; C07D 493/22; C07D 495/22; C07D 403/14

(57) ABSTRACT

A method of creating a tubular compound includes providing an end cap including a macrocycle that is sufficiently rigid that it presents a plurality of functional groups oriented axially on or extending axially from a first axial side of the end cap, creating a tubular wall from a plurality of reactive compounds which is covalently attached to the end cap via reaction of the plurality of functional groups extending from the first axial side of the end cap, the tubular wall extending axially from the first axial side end cap, and performing at least one equatorial cyclization reaction axially distant from the end cap which includes covalent bonding of residues of a group of the plurality of reactive compounds used in forming the tubular wall.

### CROSS-REFERENCE TO RELATED APPLICATIONS

This application claims benefit of U.S. Provisional Patent Application Ser. No. 63/057,506, filed Jul. 28, 2020, the disclosure of which is incorporated herein by reference.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

During the last two decades the number of applications using carbon nanotubes (CNTs) have increased dramatically. CNTs have, for example, appeared as one fundamental material to advance the construction of nanocircuits. In principle, nanocircuits could be created where the conducting and semiconducting parts are made from CNTs alone. While their conductivity properties are highly desirable, control over CNTs synthesis is poor, which manifests in properties with high batch-to-batch variability. Also, CNTs conductivity is strictly determined from their chirality. Thus, for almost the same tube diameter, conductive and semiconductive CNTs are found. Tailored syntheses to produce a unique type of CNT is challenging and currently absent in the literature. Therefore, most procedures rely on separations from complex mixtures. In short, there is a pressing need for new synthetic methods to obtain analogues of CNTs.

In that regard, the next generation of molecular-scale electronic devices will require precise control of the building blocks' electronic properties. Homogeneity of the constituents and their properties may be achieved as long as molecules are utilized instead of dispersions. Thus, fundamental nanoscience research will likely focus on controlling the main component's properties via strict control of their synthesis.

However, synthesis of macromolecules in the nanosize regime with strict control of their molecular architecture remains a challenge in the synthetic community. Preparation of such macromolecules becomes even more difficult when such macromolecules include aromatic systems in strained geometries. As a result, overcoming the buildup of strain energy has to be carefully considered during their design process. CNTs are an example of inherently strained yet stable contorted aromatics.

Before the discovery of carbon nanotubes, belt-shaped fused aromatic compounds (see FIG. 1) had been already proposed as hypothetical molecules for theoretical study. Discovery of CNTs ignited the interest of their synthesis and related CNT precursor molecules. Synthesis of the shortest possible segment making up the walls of CNTs (for example, cyclo-paraphenylenes or [n]CPPs) was first reported in 2008 almost two decades after CNTs were discovered (see FIG. 1). Many research groups across the globe have reported cycloparaphenylene-related nanorings, which are regarded as the smallest cylindrical motif comprising a given nanotube. Currently, the use of these nanobelts as seeds for the synthesis of cylindrical organic molecules has failed to deliver its initial objective. Other methodologies and a range of applications using CPPs have appeared in the literature. Despite such advances, streamlined synthesis to yield tubular systems comprised of contorted aromatic species are exceedingly rare.

Control over CNT growth has been partially accomplished by starting from preexisting units as briefly described above. These seeds usually involve 1) a short nanotube fragment, or 2) a nanotube end cap. The first method relies on cutting a preexisting isolated CNT into small pieces, treating and activating them to eventually use them as seeds for further CNT growth.

While this methodology has been reported to work successfully, it involves multiple challenging steps and the yield is rather low.

The second process uses thermally decomposed fullerenes as seeds. As described above, other similar alternatives utilize [n]cycloparaphenylenes or chemically synthesized hemispherical end caps (for example, C₅₀H₁₀). Proof-of-concept of the latter technique has been reported. However, the preparation of large quantities of such end caps is not a trivial undertaking.

Non-aromatic intermediates with sp³ carbon atoms have been used to facilitate establishing the overall connectivity before re-aromatizing the nanoring in the last step. Although this method has been successful, extending the nanoring along its main axis has proven challenging.

Thus, there remains a significant need to develop synthetic techniques to provide tubular systems/structures which may include strained aromatic species.

### SUMMARY

In one aspect, a method of creating a tubular compound includes providing an end cap including a macrocycle that is sufficiently rigid that it presents a plurality of functional groups oriented on or extending from a first axial side of the end cap, creating a tubular wall from a plurality of reactive compounds which is covalently attached to the end cap via reaction of the plurality of functional groups on the end cap extending from the first axial side thereof, the tubular wall extending axially from the first axial side of the end cap, and performing at least one equatorial cyclization reaction axially distant from the end cap which includes covalent bonding of residues of a group of the plurality of reactive compounds used in forming the tubular wall. In a number of embodiments, the macrocycle is generally cylindrical or ellipsoidal. The macrocyclic compound may, for example, include arene groups. In a number of embodiments, the plurality of functional groups on or extending from the first axial side of the macrocycle are hydroxy, alkoxy, halide or aldehyde groups. In a number of embodiments, the macrocycle is a resorcin[n]arene wherein n is an integer between 4 and 6, a bridged resorcin[n′]arene wherein n′ is an integer between 4 and 6, or a calix[n″]pyrrole-resorcinarene wherein n″ is an integer between 4 and 6.

In a number of embodiments, the plurality of reactive compounds is selected to maintain pi conjugation axially and equatorially in the tubular wall. In a number of embodiments, at least a portion of the plurality of reactive compounds include an arene group, an acene group, an N-heteroarene group or an N-heteroacene group. The tubular wall may, for example, include a plurality of acene groups, sections, or fragments or heteroatom-containing acene groups, sections, or fragments. Acenes or polyacenes are a class of organic compounds and polycyclic aromatic hydrocarbons made up of linearly fused benzene rings. Acenes, for example, have potential interest in optoelectronic applications. Additionally, conjugation in either direction, equatorial or axial, can be achieved by incorporation of acetylenic or alkynylene groups or fragments (—C≡C—) as well as alkenylene groups or fragments (—C═C—).

In a number of embodiments, the end cap includes a macrocyclic compound including arene groups and including the plurality of functional groups orients axially on or extending from the first axial side of the macrocycle and the method further includes reacting a first group of compounds of the plurality of reactive compounds with the macrocyclic compound to extend the macrocyclic compound in the axial direction. Each of the first group of compounds includes at least one functional group which is reactive with at least one of the plurality of functional groups of the macrocyclic compound extending from the first axial side thereof under a first set of reaction conditions. The first group of compounds of the plurality of reactive compounds further includes at least one of (i) at least other functional group which does not react with the plurality of functional groups of the macrocyclic compound extending from the first axial side thereof under the first set of reaction conditions and is oriented to react with one of a second group of compounds of the plurality of reactive compounds to axially extend the tubular wall, or (ii) two functional groups which do not react with the plurality of functional groups of the macrocyclic compound extending from the first axial side thereof under the first set of reaction conditions that are oriented to react to connect adjacent residues of the compounds of first group of compounds to form the at least one equatorial cyclization.

The compounds of the first group of compounds may, for example, include the at least one functional group to react with one of the compounds of the second group of compounds and two functional groups to connect adjacent residues of the compounds of first group of compounds to form the at least one equatorial cyclization. In a number of embodiments, after the compounds of the first group compounds are reacted with the macrocyclic compound, the method further includes reacting the second group of compounds with the residues of the first group of compounds to axially extend the tubular wall.

In a number of embodiments, the at least one equatorial cyclization reaction is an aryl-aryl coupling, a Suzuki-Miyaura cross coupling reaction to create a linkage including a phenylene group, a reaction to create a diimide linkage, or a reaction to create an alkynyl linkage. A plurality of equatorial cyclization reactions may, for example, be performed axially distant from the end cap.

The macrocycle may further include a second plurality of functional groups oriented axially on or extending from another or a second axial side of the end cap, opposite the first side. In a number of embodiments, the second plurality of functional groups are selected to control solubility, to participate in a polymerization reaction, or to attach the tubular compound to a substrate. In a number of embodiments, the macrocycle is:

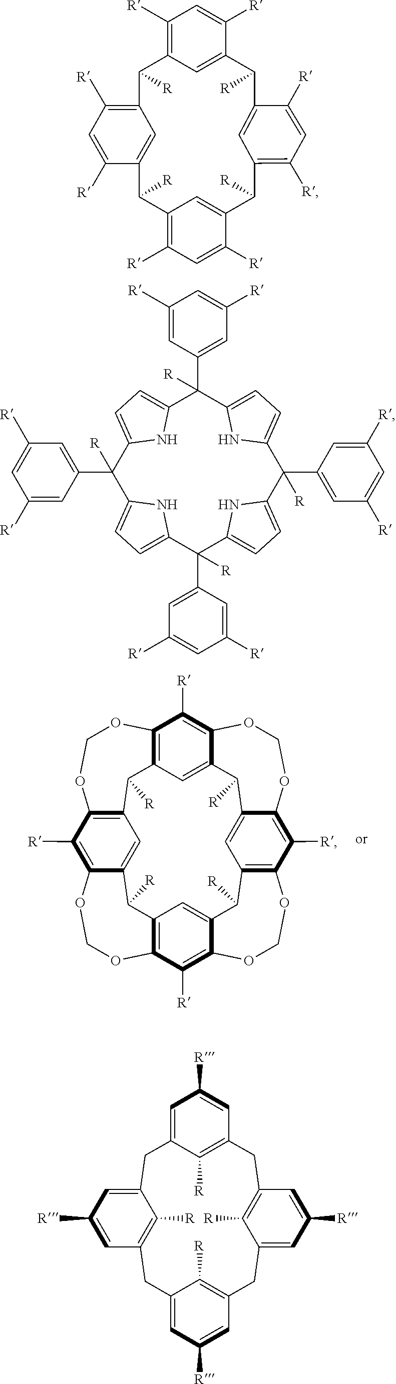

wherein R′ is a hydroxy group or an alkoxy group, R″ is a halo group or an aldehyde group, R′″ is a halo group, a hydroxy group, an amine group, an aldehyde group, or a carboxylic group, and R is an alkyl group, an aryl group, an alcohol group, an amine group, a carboxyl group, an ether group, an olefinic group, or a hydrophilic polymer. In a number of embodiment R is a functional group suitable for reaction with another functional group on a substrate or R is a polymerizable group (for example, a polymerizable olefinic group, a hydroxy group, an amine group, or a carboxyl group). Protective groups may be used in connection with certain R groups during, for example, axial extension of the tubular compounds hereof.

In another aspect, a tubular compound is formed by a process including providing an end cap including a macrocycle that is sufficiently rigid that it presents a plurality of functional groups oriented axially on or extending from a first axial side of the end cap. creating a tubular wall from a plurality of reactive compounds which is covalently attached to the end cap via reaction of the plurality of functional groups extending from the first axial side of the end cap, the tubular wall extending axially from the end cap, and performing at least one equatorial cyclization reaction axially distant from the end cap which includes covalent bonding of residues of a group of the plurality of reactive compounds used in forming the tubular wall.

In a number of embodiments, the plurality of reactive compounds is selected to maintain pi conjugation axially and equatorially in the tubular wall. In a number of embodiments, at least a portion of the plurality of reactive compounds include an arene group, an acene group, an N-heteroarene group or an N-heteroacene group. The tubular wall may, for example, include a plurality of acene groups, sections, or fragments or heteroatom-containing acene groups, sections, or fragments. Acenes or polyacenes are a class of organic compounds and polycyclic aromatic hydrocarbons made up of linearly fused benzene rings. Acenes, for example, have potential interest in optoelectronic applications. Additionally, conjugation in either direction, equatorial or axial, can be achieved by incorporation of acetylenic or alkynylene groups or fragments or alkenylene groups or fragments.

As described above, in a number of embodiments, the end cap includes a macrocyclic compound including arene groups and including the plurality of functional groups oriented axially on or extending (axially) from a first axial side thereof, and the method further includes reacting a first group of compounds of the plurality of reactive compounds with the macrocyclic compound to extend the macrocyclic compound in the axial direction. Each of the first group of compounds includes at least one functional group which is reactive with at least one of the plurality of functional groups extending from the first axial of the macrocyclic compound under a first set of reaction conditions. The first group of compounds of the plurality of reactive compounds further includes at least one of (i) at least other functional group which does not react with the plurality of functional groups of extending from the first axial side of the macrocyclic compound under the first set of reaction conditions and is oriented to react with one of a second group of compounds of the plurality of reactive compounds to axially extend the tubular wall, or (ii) two functional groups which do not react with the plurality of functional groups of extending from the first axial side of the macrocyclic compound under the first set of reaction conditions that are oriented to react to connect adjacent residues of the compounds of first group of compounds to form the at least one equatorial cyclization.

The compounds of the first group of compounds may, for example, include the at least one functional group to react with one of the compounds of the second group of compounds and two functional groups to connect adjacent residues of the compounds of first group of compounds to form the at least one equatorial cyclization. In a number of embodiments, after the compounds of the first group compounds are reacted with the macrocyclic compound, the method further includes reacting the second group of compounds with the residues of the first group of compounds to axially extend the tubular wall.

In a number of embodiments, the at least one equatorial cyclization reaction is an aryl-aryl coupling, a Suzuki-Miyaura cross coupling reaction to create a linkage including a phenylene group, a reaction to create a diimide linkage, or a reaction to create an alkynyl linkage. A plurality of equatorial cyclization reactions may, for example, be performed axially distant from the end cap.

The macrocycle may further include a second plurality of functional groups oriented on another side or a second axial side of the end cap, opposite the first side. In a number of embodiments, the second plurality of functional groups are selected to control solubility, to participate a polymerization reaction, or to attach the tubular compound to a substrate. In a number of embodiments, the macrocycle is:

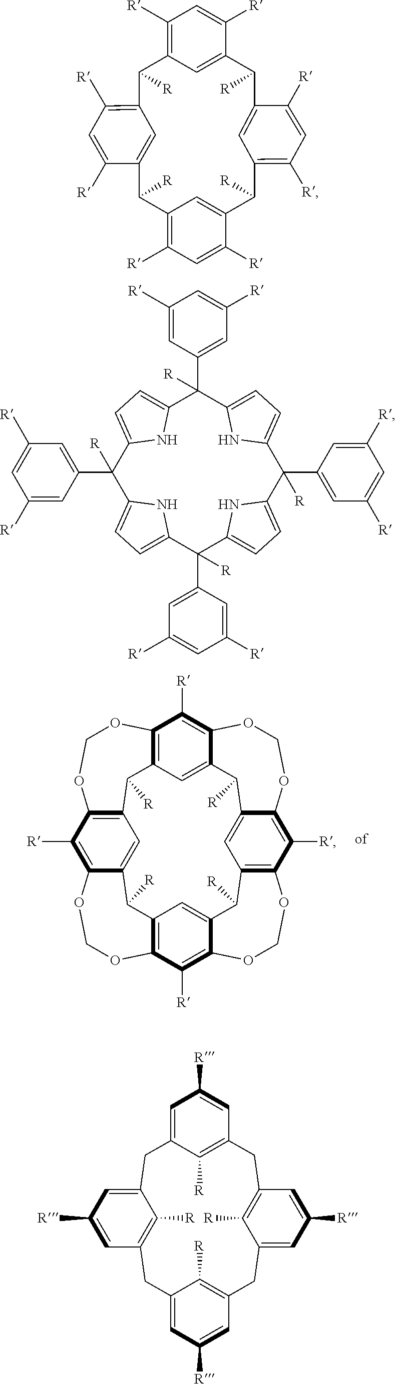

wherein R′, R″, R′″, and R are set forth above.

In a number of embodiments, the tubular compound has the formula:

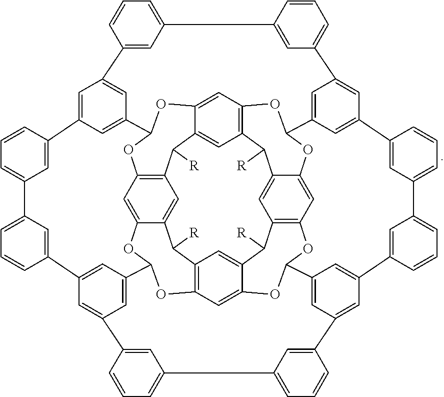

In a further aspect, a structure for use in a separation, includes tubular residues of a tubular compound formed by a process including providing an end cap including a macrocycle that is sufficiently rigid that it presents a plurality of functional groups oriented axially on or extending (axially) from a first axial side of the end cap, the macrocycle further including a plurality of functional group oriented to extend from the second axial side of the end cap, opposite the first axial side, wherein reaction of one or more of the plurality of functional groups oriented to extend from the second axial side of the end cap covalently bonds the tubular residues of the tubular compounds within the structure, creating a tubular wall from a plurality of reactive compounds which is covalently attached to the end cap via reaction of the plurality of functional groups extending from the first axial side of the end cap, the tubular wall extending axially from the end cap, performing at least one equatorial cyclization reaction axially distant from the end cap which includes covalent bonding of residues of a group of the plurality of reactive compounds used in forming the tubular wall; and reacting one or more of the plurality of functional groups oriented to extend from the second axial side of the end cap to covalently bond the tubular residues of the tubular compounds within the structure.

The one or more of the plurality of functional groups oriented to extend from the second axial side of the end cap may, for example, be reacted with one or more functional groups of a substrate for the structure in forming the structure, or one or more of the plurality of functional groups oriented to extend from the second axial side of the end cap may, for example, be reacted in a polymerization reaction in forming the structure.

As described above, in a number of embodiments, the end cap includes a macrocyclic compound including arene groups and including the plurality of functional groups oriented axially on or extending from the first axial side thereof. A first group of compounds of the plurality of reactive compounds is reacted with the macrocyclic compound to extend the macrocyclic compound axially from the first axial side. Each of the first group of compounds includes at least one functional group which is reactive with at least one of the plurality of functional groups of macrocyclic compound extending from the first axial side thereof under a first set of reaction conditions. The first group of compounds of the plurality of reactive compounds further includes at least one of (i) at least other functional group which does not react with the plurality of functional groups of the macrocyclic compound extending from the first axial side thereof under the first set of reaction conditions and is oriented to react with one of a second group of compounds of the plurality of reactive compounds to axially extend the tubular wall, or (ii) two functional groups which do not react with the plurality of functional groups of the macrocyclic compound extending from the first axial side thereof under the first set of reaction conditions that are oriented to react to connect adjacent residues of the compounds of first group of compounds to form the at least one equatorial cyclization.

The compounds of the first group of compounds may, for example, include the at least one functional group to react with one of the compounds of the second group of compounds and two functional groups to connect adjacent residues of the compounds of first group of compounds to form the at least one equatorial cyclization. In a number of embodiments, after the compounds of the first group compounds are reacted with the macrocyclic compound, the method further includes reacting the second group of compounds with the residues of the first group of compounds to axially extend the tubular wall.

In a number of embodiments, the at least one equatorial cyclization reaction is an aryl-aryl coupling, a Suzuki-Miyaura cross coupling reaction to create a linkage including a phenylene group, a reaction to create a diimide linkage, or a reaction to create an alkynyl linkage. A plurality of equatorial cyclization reactions may, for example, be performed axially distant from the end cap.

One or more of the plurality of functional group oriented to extend from the second axial side of the end cap may, for example, be reacted with one or more functional groups of a substrate for the structure in forming the structure, or one or more of the plurality of functional group oriented to extend from the second axial side of the end cap may be reacted in a polymerization reaction in forming the structure.

In a number of embodiments, the macrocycle is:

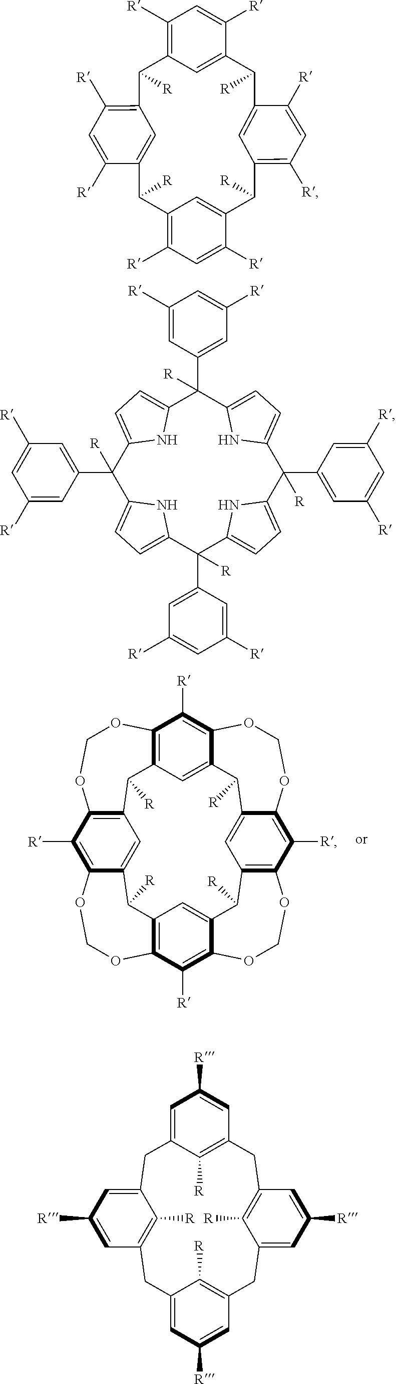

wherein R′, R″, R′″, and R are set forth above.

The tubular residue of the tubular compound may, for example, selectively interact with at least one compound to be separated from a mixture of compounds. In a number of embodiments, the tubular residue of the tubular compound selectively interacts with at least one fullerene to be separated from a mixture of different fullerenes (for example, for separation of one of a C₆₀ fullerene or a C₇₀ fullerene from a mixture including both C₆₀ and C₇₀ fullerenes).

In a number of embodiments, the tubular compound has the formula:

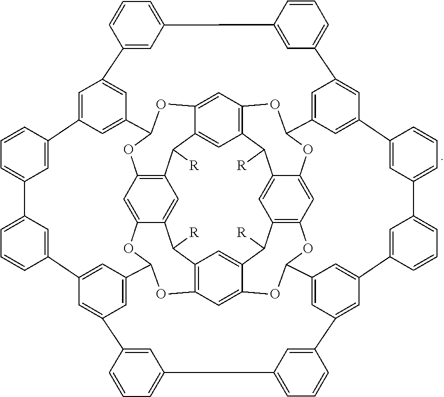

The present devices, systems, methods, and compositions, along with the attributes and attendant advantages thereof, will best be appreciated and understood in view of the following detailed description taken in conjunction with the accompanying drawings.

### BRIEF DESCRIPTION OF THE DRAWINGS

FIG. 1 illustrates armchair, zigzag and chiral carbon nanotubes and cyclic structures that can be considered short segments making up the walls of such nanotubes.

FIG. 2 illustrates schematically an embodiment of a “lego-style” or building block build up methodology for tubular compounds hereof, a representative embodiment of a tubular compound hereof, and a model of that compound.

FIG. 3A illustrates the chemical structure of a resorcin[4]arene (wherein R′ is hydroxy or aldehyde), a functionalized (wherein R″ is halo or aldehyde), methylene bridged resorcinarene, a calix[4]pyrrole-resorcinarene (wherein R′ is hydroxy or aldehyde), and another resorcin[4]arene (wherein R′″ is a halo group, a hydroxy group, an amine group, an aldehyde group, or a carboxylic group) which may be used in forming a number of embodiments of end caps hereof.

FIG. 3B illustrates representative extended structures with resorcin[4]arene and calix[4]pyrrole end caps wherein the end caps are reacted with four equivalents of 1,2-difluoro-4,5-dinitrobenzene, followed by reduction.

FIG. 4 illustrates representative building blocks which may be used in forming the walls of the tubular structures hereof.

FIG. 5 illustrates a representative embodiment of a general reaction sequence to form representative embodiments of tubular structures hereof, a top view of molecular model of compound 2a indicating the approximate diameters of the 4-(2a), 5-(2b), and 6-membered (2c) resorcin[n]arene derivatives and a side molecular model of compound 3a using diamine 14 of FIG. 4.

FIG. 6A illustrates a structure and molecular model of an embodiment of a tubular[n]arene dimer hereof which is linked together via square-planar metal coordination (linker) for use, for example, as a permanent porous liquid or PPL capsule.

FIG. 6B illustrates an embodiment of a covalent tubular[n]arene dimer formed by condensation of compound 9 of FIG. 4 (encircled fraction) with two equivalents of compound 2a

FIG. 6C illustrates doping of a tubular[n]arene hereof by metal coordination (labeled metal atoms) to phenazine fragments.

FIG. 6D illustrates an embodiment of supercapacitor construction using tubular[n]arenes hereof wherein nanoforest synthesis is achieved by covalent addition of tubular[n]arenes to, for example, amine functionalized electrodes, and wherein the porosity and redox activity of the tubular[n]arene walls permits development of supercapacitance.

FIG. 7A illustrates representative examples of tubular[n]arene precursors 16a, 17a, and 18a synthesized to include the following R groups: a—propyl, b—ethylene glycol-based groups, and c—trialkylsilyl-based groups, which have been used to study solubility.

FIG. 7B illustrates the synthesis and characterization of the precursor compound 16a, a model thereof and NMR characterization thereof.

FIG. 8 illustrates another chemical representation of an embodiment of a methodology hereof for synthesizing tubular structures.

FIG. 9 illustrates the synthesis of the final tubularene product compound of FIG. 8 and confirmation of the product compound by MALDI-TOF-MS.

FIG. 10 illustrates another embodiment of a general route to tubular structures/compounds hereof in which a benzaldehyde with bromo substituents (for equatorial cyclization) is reacted in a condensation step with an octa-amino cavitand and proton NMR characterization of the final product.

FIG. 11 illustrates characterization of the compound of FIG. 10 via MALDI-TOF.

FIG. 12 illustrates another embodiment of a general route to tubular structures/compounds hereof.

FIG. 13, panel (a) illustrates experimental MALDI-TOF MS molecular ion peaks of 1 (blue trace) and 2 (red trace), wherein the lower traces represent the simulated [M+H]+ isotopic distributions; panel (b) illustrates 1H NMR of (top) compound 21 and (bottom) compound 22 in CD₂Cl₂ at 20° C., wherein proton labels are set forth in FIG. 12; panel (c) illustrates a molecular crystal structure of 21 obtained at 150 K, wherein thermal ellipsoids are set at 50% probability level, the H atoms are omitted for clarity, and a top view of the sphere packing model (van der Waals radii) of 21; and panel (d) illustrates DFT calculated barrier for ring flipping, wherein rotation of the phenyl moiety was followed by tracking the dihedral angle between the highlighted carbon atoms.

FIG. 14 illustrates HOMO (highest occupied molecular orbital) and LUMO (lowest unoccupied molecular orbital) density plots (±0.02 au) of: panel (a) compound 21 and panel (b) compound 22; panel (c) illustrated UV-Vis absorption (solid trace) and emission spectra (dotted trace) of 21, 22, and 23, collected in CH₂Cl₂ at room temperature; panel (d) illustrates cyclic (CV) and differential pulse voltammetry (DPV) for 21, panel (e) illustrates cyclic (CV) and differential pulse voltammetry (DPV) for 22, in ortho-dichlorobenzene at room temperature, wherein CV scan rate: (d) 100 mV/s, and (e) 50 mV/s; A 0.1 M [n-Bu₄N][PF₆] solution was used as supporting electrolyte, the labels on italics correspond to the oxidation level q represented as: (d) [21]q, and (e) [22]q, the E½ potentials were obtained from the DPV data and DPV traces are shifted down for better visualization of the data; and panel (f) illustrates experimentally determined HOMO-LUMO energy levels of 21, 22, and [8]CPP.

FIG. 15 illustrates another embodiment of a general route to tubular structures/compounds hereof in which an Octa-Br end cap or template (Octa-Br template) is reacted with a cross-coupling agent.

FIG. 16 illustrates another embodiment of a general route to tubular structures/compounds hereof in which another Octa-Br end cap or template (Octa-Br template 2) is reacted with a cross-coupling agent.

FIG. 17 illustrates another embodiment of a general route to tubular structures/compounds hereof in which another Octa-Br end cap or template (Octa-Br template 3) is reacted with a cross-coupling agent.

FIG. 18 illustrates another embodiment of a general route to tubular structures/compounds hereof in which another Octa-Br end cap or template (Octa-Br template 4) is reacted with two different cross-coupling agents.

FIG. 19 illustrates products (top rim connectivity only) of the reaction of FIG. 17.

FIG. 20 illustrates a shift in the fluorescence from blue-to-red (from left-to-right) of isolated products set forth in FIG. 19.

FIG. 21 illustrates another embodiment of a general route to tubular structures/compounds hereof in which end caps or templates octa-chloro 1 and octa-chloro 2 undergo an equatorial cyclization to form compounds nanotube1 and nanotube2, respectively.

FIG. 22 illustrates that isolated produce nanotube1 of FIG. 21 displays host-guest binding properties towards fullerenes C₆₀ and C₇₀.

### DETAILED DESCRIPTION

It will be readily understood that the components of the embodiments, as generally described and illustrated in the figures herein, may be arranged and designed in a wide variety of different configurations in addition to the described representative embodiments. Thus, the following more detailed description of the representative embodiments, as illustrated in the figures, is not intended to limit the scope of the embodiments, as claimed, but is merely illustrative of representative embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “a compound” includes a plurality of such compounds and equivalents thereof known to those skilled in the art, and so forth, and reference to “the compounds” is a reference to one or more such compounds and equivalents thereof known to those skilled in the art, and so forth. Recitation of ranges of values herein are merely intended to serve as a shorthand method of referring individually to each separate value falling within the range. Unless otherwise indicated herein, and each separate value, as well as intermediate ranges, are incorporated into the specification as if individually recited herein. All methods described herein can be performed in any suitable order unless otherwise indicated herein or otherwise clearly contraindicated by the text.

In a number of embodiments hereof, macrocycles are used to template the synthesis of strained conjugated aromatics. In representative embodiments, the methodologies hereof may be extended to the synthesis of longer and more complex nanotube structures. As described further below, the compositions hereof may also be used, for example, in the formation of permanently porous liquids or PPLs (for example, for used in alkane/alkene gas separations), in forming container-shaped molecules for use in, for example, host-guest chemistry, in the formation of seeds for nanotubes, in the formation of nanoelectric devices such as supercapacitors and in other uses. The highly strained, aromatic architectures hereof, which have a tubular shape, are sometimes referred to herein as tubularenes as initial embodiments of such tubular structures were based on resorcin[n]arenes or upon calix[n]pyrrole-resorcinarenes.

As used herein “tubular” refers to an extending structure which is hollow or has an internal void. In a number of embodiments, the tubularenes hereof are conjugated atomically-precise cylindrical organic architectures that may, for example, be used as alternatives to CNTs or seeds for growing CNTs. In a number of embodiments, it is desirable that the tubular structures are as symmetrical as possible in cross-section over the axial length thereof. In a number of embodiments, the tubular structures are generally cylindrical.

In representative methods hereof, molecular precursors or end caps are first designed and synthesized, to obtain conjugated, atomically-precise cylindrical or tubular organic architectures or tubular[n]arenes. In that regard, the tubular structures hereof may be constructed through a bottom-up, Lego-like (or building-block) assembly of relatively small molecules or assemblies thereof of predetermined size, functionality and conformation which are used in the manner of building blocks. The predetermined size, functionality and conformation of the building blocks provides for controlled reactive, covalent assembly. In embodiments wherein conductive properties are of importance, the tubular[n]arene's electronic structure may be tailored by, for example 1) constructing them using a variety of electronically suitable building blocks (pre-synthesis), and 2) by post-synthetic covalent manipulations and/or metal coordination. In general, if electrical properties such as conductivity are important, it is desirable to maintain pi conjugation throughout the tubular wall of the tubular structure hereof (both axially and equatorially). The tubular[n]arene's electronic and ionic charge capacity properties may be readily characterized and their assembly into various devices may be readily explored.

Multi-wall CNTs have penetrated academic and commercial settings, where such CNTs find use in a wide range of applications such as solar cells, supercapacitors, coatings and films, batteries, composite materials, water filters, biosensors, among many more. All the latter examples use CNTs as polydisperse materials. Realizing strict control of size (diameter) and conductivity (chirality) promise to revolutionize the field of nanoelectronics. As described above, however, such control remains a significant challenge. Developing analogous wire-like compounds as described herein presents an attractive and potentially very rewarding avenue to explore.

Currently, there exists no analogues to carbon nanotubes. By developing synthetic approaches to conjugated nanotubes (either conducting and/or semiconducting) or molecular wires, compositions hereof may expand and improve the toolbox of electronic materials for nanodevices. The synthetic methods described herein are modular and tunable to hone the resulting nanotube for a particular application.

For example, CNT-based supercapacitors have been explored as high energy and power density devices. Such supercapacitors hold great promise in terms of durability. However, to realize them, CNTs have to be forest-grown in the electrode material. For best performance, such growth must occur as uniformly as possible. However, this growth remains a significant challenge in the construction of those devices. By introducing functionality into the nanotube, as possible in the tubularenes described herein, one may build uniform and controlled nanoforests with electroactive properties for supercapacitor applications.

Carbon allotropes such as graphene and CNTs have also had an important impact redefining new standards in materials properties. Alternative materials forming single layer 2D systems are currently under exploration to compete with graphene. Once again, however, no analogues exist for CNTs. Creating analogues of CNTs represents a large untapped potential for new materials discovery and/or development. For example, the methods hereof may be used in the synthesis of fluid, capsule-like molecules which may be used as separators of complex alkane/alkene mixtures. Such separations currently employ energy intensive cryogenic distillations.

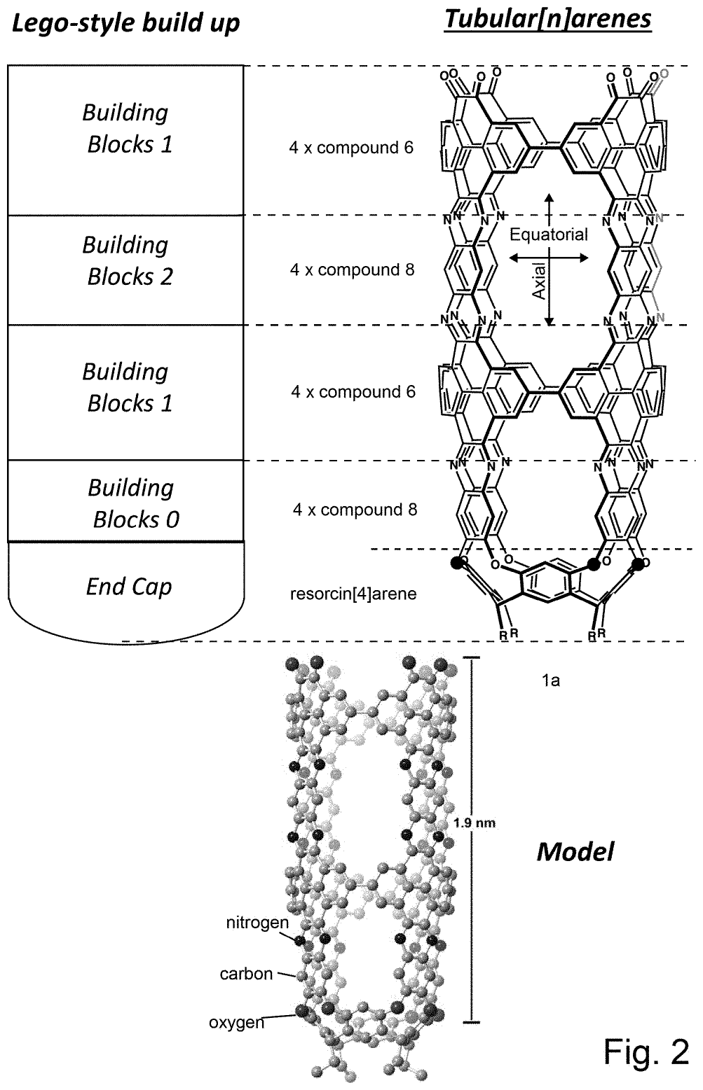

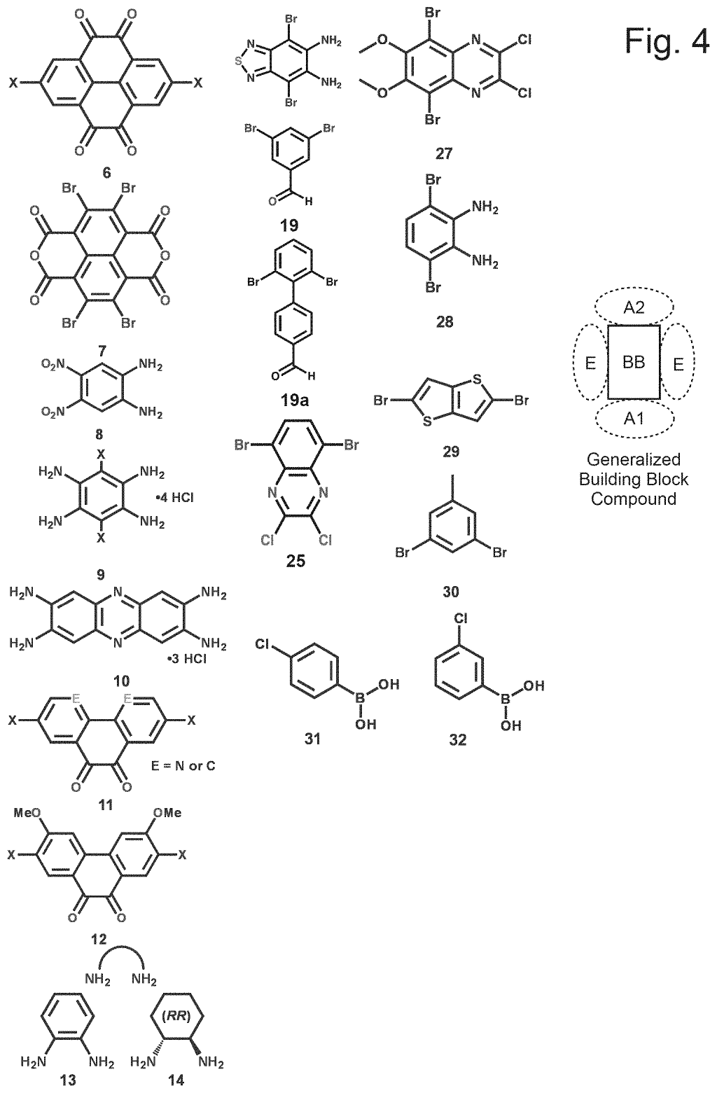

Representative examples of tubular structures hereof with conductive properties are illustrated, for example, in FIG. 2. In a number of embodiments, the synthetic approach is based on a step-by-step growth—also referred to as a “Lego-style” or a building-block build up. Two general methods are, for example, possible. The first method or synthetic route starts directly from the end cap to facilitate the cylindrical growth right at the beginning (sometimes referred to as divergent synthesis). Alternatively, in a second method or synthetic route, sections of the nanotube walls are preformed and later reacted (for example, condensed) with the end cap (sometimes referred to as a convergent approach). Using a convergent approach, large precursor fragments of the tubularene wall are made separately, using two or more building blocks, and condensed into the end cap or macrocycle towards the end of the reaction sequence. Such fragments may be generally linearly extending. Such a linear extending fragment may, for example, be synthesized by reacting compound 6 (see FIG. 4) with compound 8, and then reacting the resultant compound with compound 6 to provide a length of wall as illustrated in FIG. 2. Alternatively, such a compound can be synthesized by aryl coupling of two compounds 6 and reaction of the resultant compound with one or two of compound 8 to form a portion of the wall as illustrated in FIG. 2. In general, yields may be higher with a convergent synthetic approach.

The formation of the tubular frameworks described herein is dependent on the end cap or macrocycle. In a number of embodiments hereof, the molecule forming the basis of the end cap should have two basic features: 1) it should be (semi)rigid and cyclic to translate its shape into a tubular or generally cylindrical geometry; and 2) it should contain free functional groups oriented into one (axial) direction to allow for preferential growth along this direction of the axis. In general, the end-cap or macrocycle should be sufficiently rigid so that, after accounting for its intrinsic degrees of freedom, it presents its functional groups oriented all in the same axial direction. Several macrocyclic compounds suitable for use herein are illustrated in FIG. 4A. In terms of rigidity, resorcin[n]arene is more rigid than, for example, calix[n]pyrrole-resorcinarene, simply because the latter compound has more degrees of freedom that arise from the single bonds which allow the resorcinol fragment to rotate.

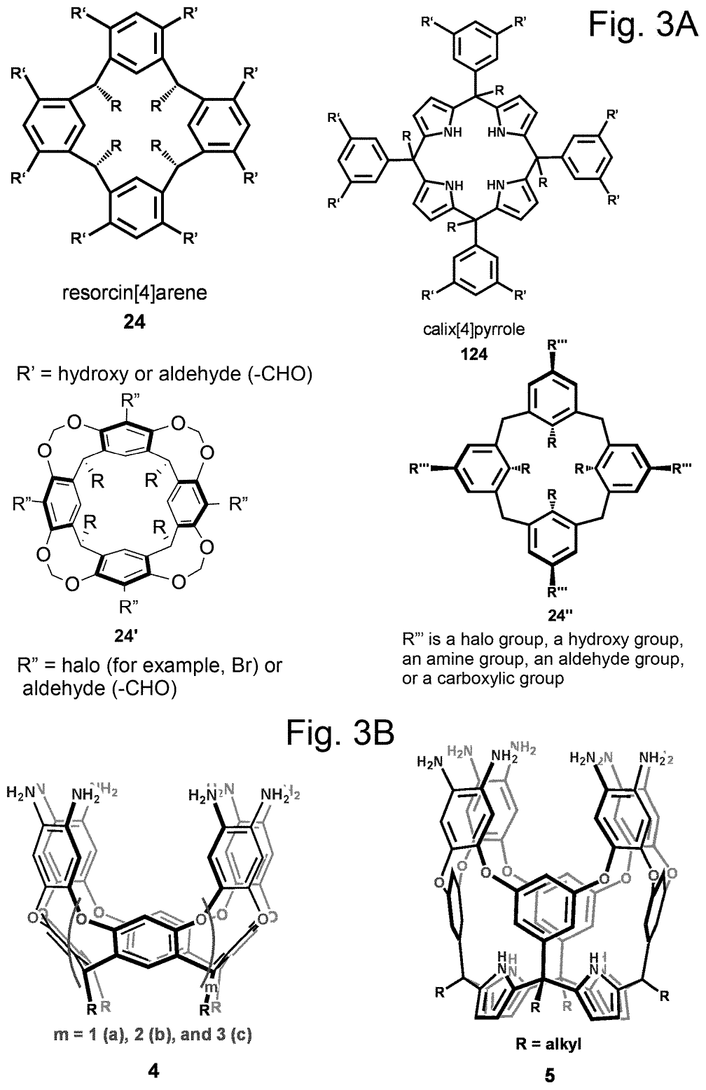

As described above, two representative, general families of compounds fulfill those requirement. The first general family of compounds is the resorcin[n]arene family of compounds as described above wherein macrocycles are built from condensation of resorcinol and an aldehyde. The second family of compound is the calix[n]pyrrole-resorcinarene family of compounds. Such families are highly modular since they allow the end group, R (as illustrated in FIG. 3A) to vary over a wide variety of substituents. See, for example, Timmerman, et al., “Resorcinarenes”, Tetrahedron, 52:8, 2663-1704 (1996), the disclosure of which is incorporated herein by reference. In a number of embodiments, the group R is an alkyl, an aryl, an alcohol, an amine, a carboxylic group, or an ether group. In a number of embodiment R is a hydrophilic polymer or oligomer. In general, within the same molecule the R group is the same. However, one can have mixtures of molecules with different R groups by making such molecules separately and later mixing the pure compounds. The groups R may, for example, include or be functional groups that allow attachment of the tubularene structures/compounds here to a surface or substrate via functional groups on the surface or substrate which are reactive with the functional group of the groups R. Moreover. R groups may be chosen to allow polymerization of the tubularene structures/compounds hereof. The polymerization process may, for example, be via a radical mechanism, anionic or cationic mechanisms, or condensation reactions. Condensation polymerizations may, for example, be carried out in the case that R includes or is a hydroxyl group, an amine group, or a carboxylic acid group. In a number of embodiments, the R group is an olefinic group suitable for undergoing a radical polymerization. The radical polymerization may, for example, be a free radical polymerization or a controlled/living radical polymerization.

The terms “alkyl”, “aryl” or and other groups refer generally to both unsubstituted and substituted groups unless specified to the contrary. Unless otherwise specified, alkyl groups are hydrocarbon groups and are preferably C2 to C12 (that is, having 2 to 12 carbon atoms) alkyl groups, saturated and unsaturated (that is containing double bonds or not), and can be branched or unbranched, acyclic or cyclic. The above definition of an alkyl group and other definitions apply also when the group is a substituent on another group (for example, an alkyl group as a substituent of an alkylamino group or a dialkylamino group). The term “aryl” refers to phenyl or naphthyl, which may contain electron donating and withdrawing functional groups like carboxylic acid, esters, ethers, amines, where these functional groups may be substituted with alkyl groups as defined above. As used herein, the terms “halogen” or “halo” refer to fluoro, chloro, bromo and iodo. Heteroaryl/heteroarene groups may contain one or more heteroatoms such as N, O, S and P.

The term “amine” refers to the group —NR^{a}R^{b}, wherein R^{a} and R^{b} are for example, independently hydrogen, an acyl group, an alkyl group, and an aryl group. The term “alcohol” refers to the group —R^{c}, wherein R^{c} is, for example, an alkyl, aryl group, and a polyether which contains a free hydroxyl at the terminal or internal position of the overall fragment. The term “ether” refers to —R^{d}OR^{c} wherein R^{d} and R^{e} are, for example, independently alkyl and aryl as defined above.

Hydrophilic oligomers or hydrophilic polymers may, for example, be selected from the group consisting of hyaluronic acid, glucan, chitosan, a polyalkylene oxide, a polyvinylalcohol, a polyacrylic acid, a polyacrylamide, a polyoxazoline, a polysaccharide and a polypeptide. In a number of embodiments, the at least one hydrophilic polymer is a polyalkylene oxide. The polyalkylene oxide may, for example, be a polyethylene glycol. A polyethylene glycol or other hydrophilic polymer hereof may, for example, have a molecular weight in the range of 200 to 2000 Da in a number of embodiments.

The groups set forth above, can be substituted with a wide variety of substituents. For example, alkyl and other groups may be substituted with a group or groups including, but not limited to, a benzyl group, a phenyl group, a hydroxy group, an amino group (including, for example, free amino groups, alkylamino, dialkylamino groups and arylamino groups), and halo groups.

As used herein, the term “polymer” refers to a chemical compound that is made of a plurality of small molecules or monomers that are arranged in a repeating structure to form a larger molecule. Thus, a polymer is a compound having multiple repeat units (or monomer units) and includes the term “oligomer,” which is a polymer that has only a few repeat units. The term “copolymer” refers to a polymer including two or more dissimilar repeat units (including terpolymers—comprising three dissimilar repeat units—etc.). Polymers may occur naturally or be formed synthetically. The use of the term “polymer” encompasses homopolymers as well as copolymers. The term “copolymer” is used herein to include any polymer having two or more different monomers. Copolymers may, for example, include alternating copolymers, periodic copolymers, statistical copolymers, random copolymers, block copolymers, graft copolymers etc. Examples of polymers include, for example, polyalkylene oxides such as polyethylene glycol.

The number of aromatic units in the macrocycles or end caps hereof can be altered. For example, recent reports describe high-yielding syntheses for the 5- and 6-membered resorcin[n]arenes. Those compounds allow variability in the diameter of tubular structures hereof (see, for example, FIG. 3B). Calix[4]pyrrole-resorcinarene derivatives (compound 124 in FIG. 3A), comprise another family of end caps as described above. Calix[4]pyrrole resorcinarenes have been reported recently as analogues of resorcin[n]arenes. These families of compounds share the common feature of a rigid backbone cap and directed functional groups. Also, calix[4]pyrrole-resorcinarene compounds present the added feature of including a metal coordination site at its base, (that is, the tetrapyrrole coordination site). While the R groups on the resorcin[n]arenes are oriented collinearly with the nanotube main axis, the R groups in the calix[4]pyrrole-resorcinarene are orthogonal to this axis. The above templates offer an advantages in synthetic viability, tunability, and selectivity over previously used fullerene-, corannulene-, or [n]cycloparaphenylene-based seeds or end caps.

The methodologies hereof, as, for example, illustrated schematically in FIG. 2, represent viable and tunable methodologies to synthesize atomically precise, cylindrical or tubular organic structures or tubular[n]arenes. As described above, the Lego-style or building-block-style, build-up approach may, for example, take advantage of well-known resorcin[n]arenes as a representative template in the formation of the cylindrical or tubular structures upon stepwise condensations of building blocks 0, 1, 2 . . . and so on. The present methodologies enable control of the length and diameter of the resultant conjugated tubular nanomolecules. Moreover, the methodologies hereof benefit from the use of inexpensive and commercially accessible chemicals. Alternatively, the chemical building blocks can be synthesized in multigram scale. In a number of embodiments hereof, a foundation for the desired molecules (for example, a resorcin[n]arene) provides a cyclic template (cap or end cap) for installing the subsequent building blocks and may control the solubility profile of the final product via, for example, selection of the R substituents thereof (that is, substituents on the axial side thereof opposite the side upon which the nanotube is extended).

Representative compound 1a in FIG. 2 may be referred to as a tubular[4]arene wherein the numeral 4 is derived from the underlying resorcin[n]arene used in forming the end cap. Once again, the formation of the tubular architecture is facilitated by the use of well-known macrocycles such as resorcin[n]arenes, functionalized, methylene bridged resorcin[n]arenes, or calix[n]pyrrole-resorcinarenes (as illustrated in FIG. 3A, wherein n=4) as growth templates or end caps. There is abundant literature on those and related macrocycles that may also be used in the methodologies hereof. The walls of the tubular[n]arenes hereof may, for example, include N-heteroacene sections, fragments, or residues by design. In that regard, N-heteroacenes are known to have record high electron mobilities within the field of organic semiconductors.

Representative extended or modified end caps formed with resorcin[4,5 and 6]arene 4a, 4b and 4c and calix[4]pyrrole 5 are illustrated in FIG. 3B. Compound 4a (an octa-amino cavitand) was, for example, used in a number of representative studies hereof as the templating seed for tubular[4]arene growth. Compound 4a is produced by the reaction of 1,2-difluoro-4,5-dinitrobenzene (compound 8 in FIG. 4) with resorcin[4]arene, and subsequently reduced with hydrogen using Pd/C. Upon reaction, there is further reduction of the dinitrobenzene to form a diamine. End caps hereof may also be formed with the 5- and 6-membered resorcinarenes 4b and 4c, respectively. Employing, for example, standard Schiff base formation reactions and Ar—Ar coupling, one can accomplish a step-by-step growth to go from 4a and 6 to tubular species as illustrated in FIG. 2. In general, growth starts from the end cap and step-by-step addition of the building blocks extends the length of the tubular[n]arene. Various representative compounds/building blocks for use in forming the tubular structures hereof are illustrated in FIG. 4.

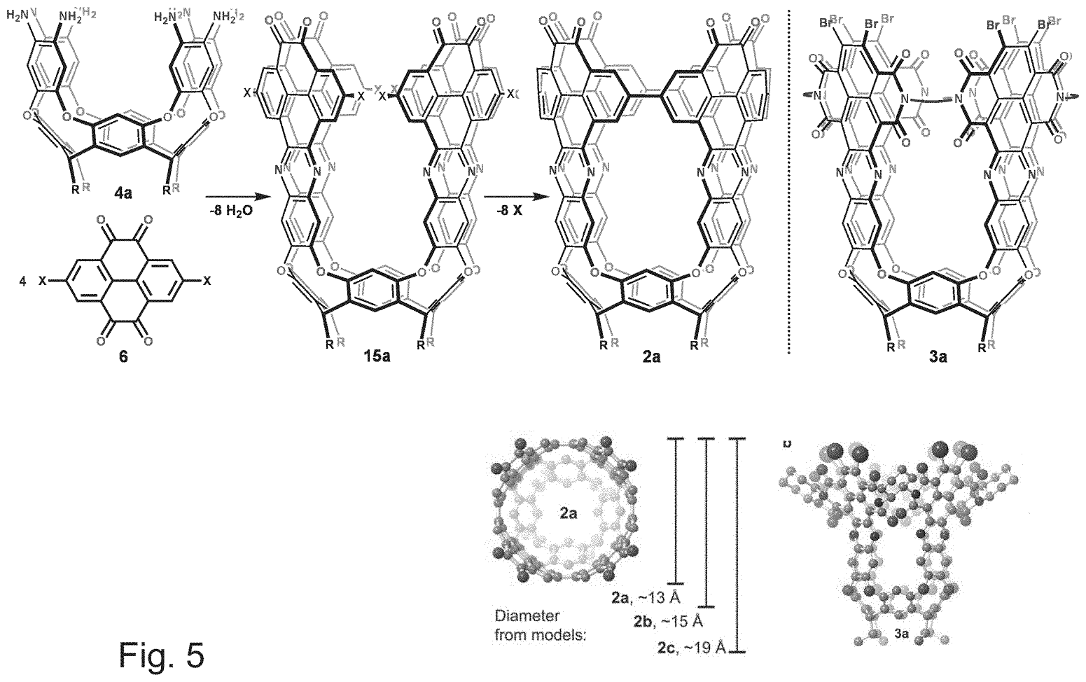

An example of a general reaction sequence to obtain the tubular[n]arene 2a is illustrated in FIG. 5. Tubular[n]arene 3a of FIG. 5 exemplifies a different wall construction where the diimides may enhance the overall redox activity of the nanotube.

As described above, the macrocyclic compounds, end caps or templates hereof are generally cylindrical and include arene groups in a number of embodiments. The macrocyclic compounds further include a plurality of functional groups oriented generally axially on one axial side of the macrocyclic compound. In a number of embodiments, the plurality of functional groups of the macrocycle is reacted with a first group or plurality of compounds or building blocks (for example, building blocks/compound groups 0, 1 and 2 of FIG. 2) to extend the macrocyclic compound in the axial direction. In a number of embodiments, each of the first group of compounds is identical. As exemplified by compound 8 used in forming extended structure 4n, in a number of embodiments, each of the first group of compounds includes two functional groups on an arene group thereof which are reactive with two of the plurality of functional groups of the macrocyclic compound (such as resorcin[n]arenes 24 and calix[n]pyrrole-resorcinarenes 124) under a first set of reaction conditions. In a number of embodiments, the two functional groups of the first group of compounds react via an aromatic nucleophilic substitution with the plurality of functional groups of the macrocyclic compound under the first set of reaction conditions. In the case of functionalized, bridged resorcin[n]arene 24′ a single functional group is presented for reaction with each of the first group of compounds.

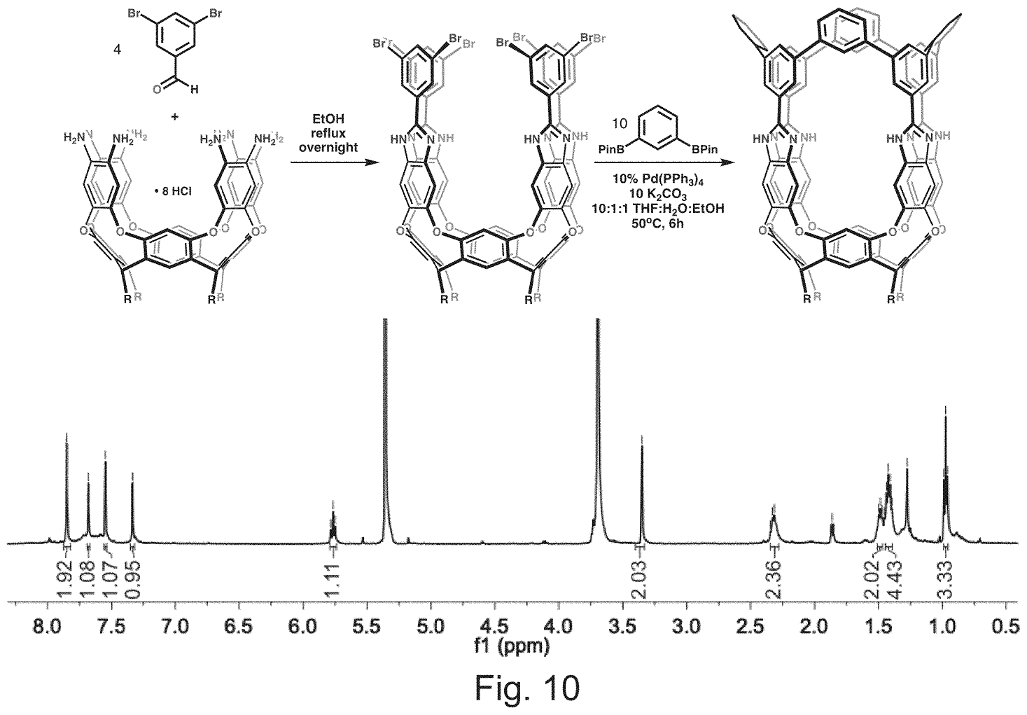

In a number of embodiments, reactions with the halo groups include two general categories: (i) cross coupling reactions (see, for example, FIG. 10) and (ii) lithium-halogen exchange followed by a substitution reaction, where the lithiated species acts as the nucleophile. In a number of embodiments, axially extending reactions with an aldehyde are condensation reactions with either one or two primary amines. An example of a condensation with two primary amines is shown in FIG. 10. Reactions of an aldehyde with a single primary amine will result in the formation of imines.

The compounds of the first group of compounds further include one or more other functional groups which do not react with the plurality of functional groups of the macrocyclic compound under the first set of reaction conditions. After each of the first group of compounds is reacted with the macrocyclic compound, the one or more other functional groups may be oriented either to react with another group of compounds or building blocks hereof to extend the structure axially (as, for example, in compound 8 of building blocks/group 0 in the embodiment of FIG. 2 and in FIG. 3B) or to react in an equatorial cyclization in which adjacent residues of the first group of compounds are connected (as, for example, in compound 25 of the embodiment of FIG. 12). As used herein, the term “equatorial” refers to elements extending in a direction essentially in the plane of the cyclic, tubular structure of the tubular compositions hereof. In cases wherein the one or more other functional groups are be oriented to react in an equatorial cyclization, two such functional groups are present. In cases where the one or more other functional group are oriented to axially extend the structure, one or two of such functional groups are present. The first group of compounds may include both one or more function groups to react with a compound of a second group of compounds or building blocks hereof to extend the structure axially and two functional groups to react in an equatorial cyclization. The compounds of other groups of building blocks used in forming an extending cylindrical structure such as illustrated in FIG. 2 are also functionalized for axial extension and/or equatorial cyclization.

Once again referring to the embodiment of FIG. 2, a generally cylindrical wall is covalently attached to and extends axially from the end cap. The wall is created, formed or assembled via the covalent connection of a plurality of controllably reactive, functionalized molecules or building blocks. In the illustrated embodiment, after formation of the extended end cap 4a, individual pluralities or groups of such plurality of reactive molecules (for example, building blocks/groups 0, 1 and 2 of FIG. 2) form sections of controlled length and functionality as the wall is extended (and eventually ended). In general, at least one equatorial cyclization reaction is carried out axially distant from the end cap (above the end cap in the orientation of FIG. 2) wherein covalent attachment occurs between adjacent residues/compounds. A plurality of such equatorial cyclization reactions, axially spaced along the axial length of the tubular wall, may be carried out as further illustrated in FIG. 2.

In addition to illustrating representative building block compounds hereof, FIG. 4 also illustrates a generalized building block compound hereof. In general, all building block compounds (that is, compounds used to extend the tubular structures hereof include at least one functional group A1 which is reactive with one or more generally axially upward oriented (in, for example, the orientation of FIG. 2) functional groups of the compounds of the group below (or the end cap) under a defined set of reaction conditions. In a number of embodiments, each block of building block compounds (building blocks 0, building blocks 1, and building blocks 2 in FIG. 2) represents a group of like or identical compounds (four compounds in the illustrated embodiment of FIG. 2) which are reacted with the extended end cap or with the group below. The building block compounds may further include two functional groups E which are oriented to connect adjacent compounds/residues within the group to create an equatorial cyclization. In general, such functional groups are different from the functional group or groups A1 and may react under a different set of defined reaction conditions. Finally, each building block compound may also include one or more functional groups A2 which are oriented to further axially extend the tubular structures hereof by reaction with a building block compound of another group of building block compounds. As clear to one skilled in the art, a rational, sequential buildup of, for example, compound 1a and other tubularenes hereof is readily achieved by selection of suitable building block compounds of predetermined functionality/reactivity.

Compound 8 of FIG. 4 sets forth a representative example of a compound including functional groups A1 and A2. Compound 6 of FIG. 4 sets forth a representative example of a compound including functional groups A1, A2 and E. In each of compounds 6 and 8, A1 and A2 include two functional groups. As illustrated in FIG. 2 and discussed further below, a Schiff base condensation reaction of the two ketone groups of compounds 6 with the two amine groups of a residue of compound 8 results in a N-heterocyclic arene to axially extend the structure, providing rigidity and electron mobility. Compound 19 provides an example, of a compound with a single reactive group A1 (an aldehyde group) and two functional groups E (bromine groups).

The core or BB (see, the generalized formula of FIG. 4) of the building block compounds hereof may, for example, include an arene or a heteroarene group, a plurality of coupled arene or heteroarene groups or fused arene or heteroarene groups as illustrated in FIG. 4. Once again, such arene-containing or arene-based compounds provide for both rigidity and conjugation. In the case the electrical properties of the compounds or compositions hereof are not of importance (for example, in PPL), building block which do not include arene groups may be used. However, such groups are preferably selected to maintain rigidity. For example, building blocks including nonaromatic cyclic compounds and/or other relatively rigidly bonded (for example, pi-bonded) components may be used. Even when electrical properties are not of importance, aromatic groups may be desirable in the compositions hereof for rigidity and for interaction with, for example, a species to be hosted or separated.

In general, building blocks such as the representative building blocks of FIG. 4 allow one to construct step-by-step nanotubes with, for example, conjugated (providing electron delocalization) and redox active walls (allowing the addition of external electrons). Such compounds also present heteroatoms, in the form of, for example, pyrazine rings or bipyridine sites, which may, for example, be used as coordination sites as discussed further below. Covalent modifications can be performed post-synthesis on some of these building blocks (for example, alkylation of compound 10), which can drastically change the electronic structure of the synthesized nanotube. Alkylation of these pyrazine rings can, for example, introduce cationic radical nature to the overall system, thus potentially increasing its conduction properties as a result of delocalization of the radical.

Once again, in a number of embodiments hereof, programmed tubular growth may commence from resorcin[n]arene-based end cap such as compound 4a. The axial projection of the diamines predisposes the nanotube wall growth into one direction as, for example, illustrated in FIG. 5 in which reaction of extended end cap 4a with building block/compound 6 (wherein, X of compound 6 is a halo atom) is illustrated. Schiff base condensation of such amines with the corresponding diketone fragment produces a pyrazine aromatic ring, as in compound 15a of FIG. 5. Relatively, short tubular[n]arenes such as compound 2a are obtained after an equatorial cyclization via, for example, aryl-aryl coupling (for example, Ullman coupling). The walls of the tubular[n]arenes hereof may have rich optical and electrochemical properties based on similar isolated organic fragments reported in the literature. An alternative approach to aryl-aryl coupling involves the condensation of the end cap with naphthalene dianhydride derivatives such as compound 7. Species synthesized in this manner (for example, compound 3a), allow the addition of external functionality via the diamine backbone. Compound 3a may, for example, be synthesized from compound 4a and compound 7 (controlling the reaction to prevent polymerization). The product of that reaction is mixed with a diamine to enforce diimide formation. Alternative routes include the formation of the diimide with a small amine (low boiling point) prior to the substitution reaction. Aside from serving as structural elements, chiral cis-diamines 13 and 14 offer novel optical properties to the tubular[n]arene.

Once again, the nanotube diameter is controlled by the end cap. Molecular models of compound 2a-c (4-membered (2a), 5-membered (2b), and 6-membered (2c) resorcin[n]arene derivatives) set forth diameters ranging from ˜13 to ˜19 Å, respectively (see FIG. 5). In contrast to the more cylindrically symmetric molecules produced from compounds 2a-c, the molecular model of compound 3a (using compound 14 in the equatorial reaction; and also being illustrated in FIG. 5), presents a widening of the outer rim's diameter. With these tubular species, which include axially oriented functional groups, growth may be continued in the lego-style manner described herein.

It is well-known that N-heteroacenes have a rich redox chemistry. By extension, tubular[n]arenes may display enhanced redox behavior since their walls may include multiple units of such heteroacenes conjugated. The charged states of such tubular structure may be determined by electrochemical methods as known in the art. These determinations, in turn, may be used to guide efforts of, for example, metal doping by performing stoichiometric chemical reductions. It is expected that the tubular[n]arene's conductivity properties will develop as the presence of itinerant or delocalized electrons is induced. Once may, for example, fine tune the end properties to form molecular wires.

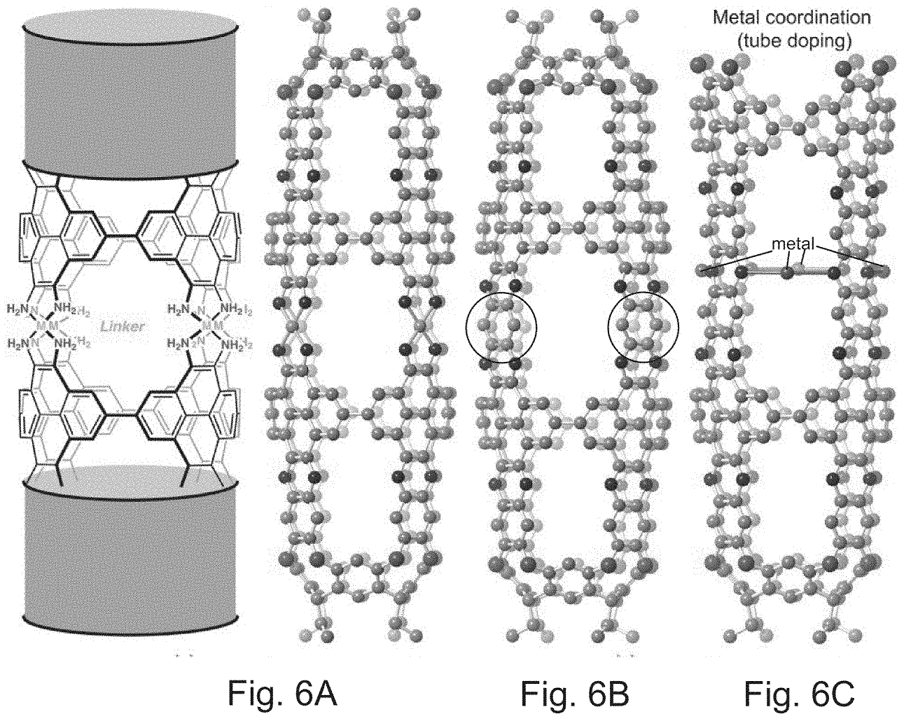

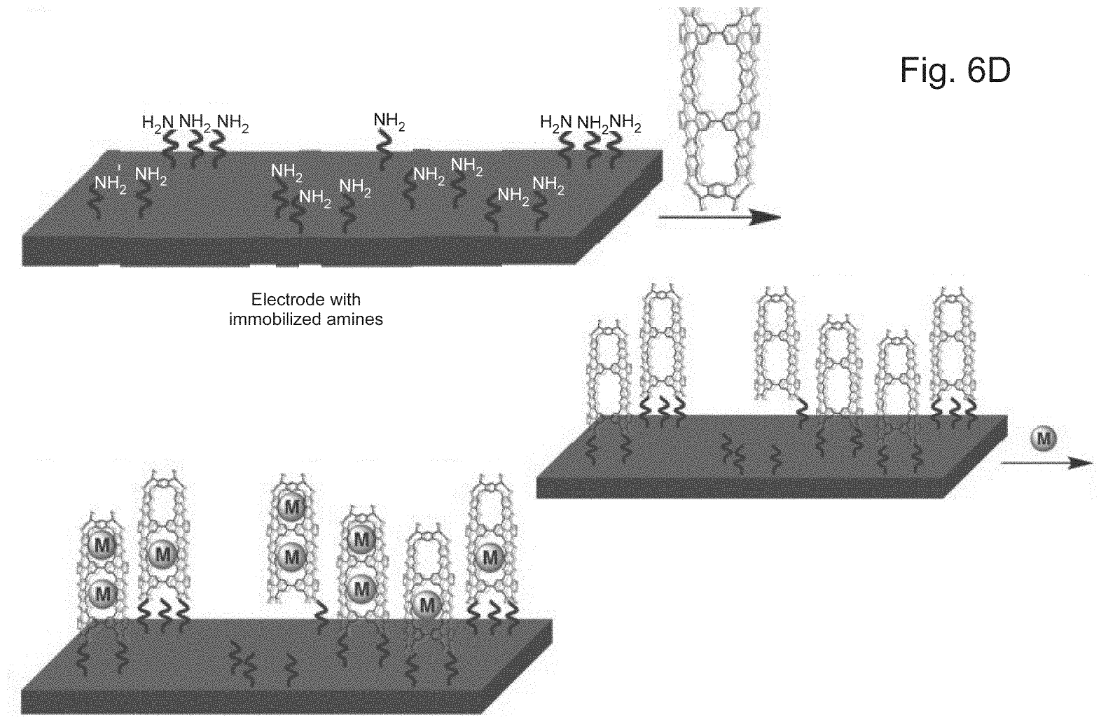

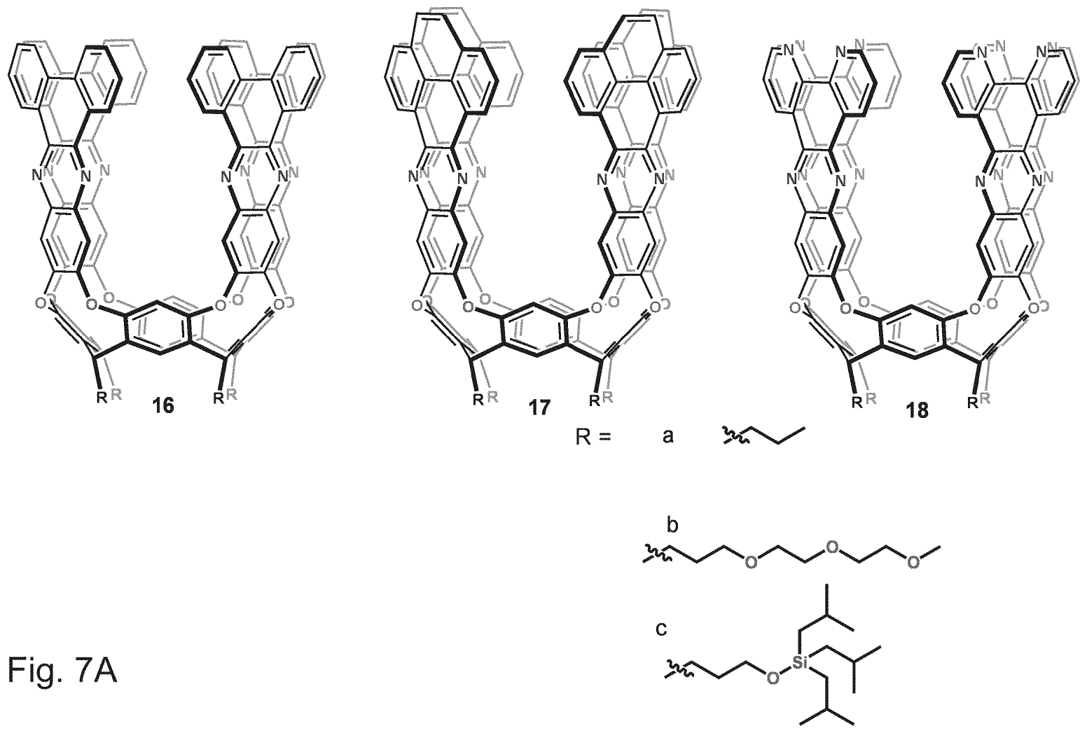

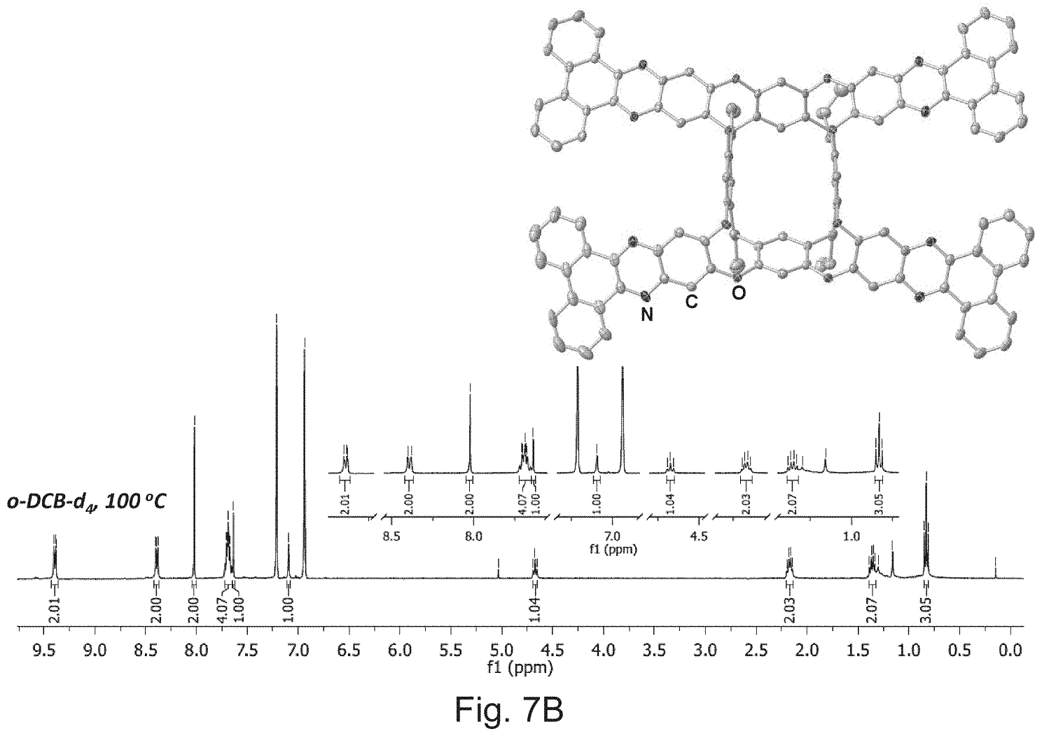

Tubular[n]arenes or tubular structures hereof are a new family of organic compounds. As briefly described above, there are many potential applications for such compounds. As illustrated in FIGS. 6A and 6B, rigid dimer assemblies (of compound 2a) may provide rigid capsules with an unrivaled interior empty space. These capsules, with the proper R groups on compound 2a, may become liquids. Such R groups may, for example, be polyalkylene oxides (for example, polyethylene oxides), as the one shown in FIG. 7A, or long chain branched alkanes. For example, studies on the synthesis of compound 4a with diethylene glycol monomethyl ether chains on the R position indicate that at medium temperatures (that is, 60-100° C.) these behave as fluids. Porous liquids have been proposed since 2007 and just recently realized. An important benefit of porous liquids is that they enhance the solubility of small hydrocarbon gases, such as CH₄. Thus, exploring fractional gas separations between closely related molecules such as CH₄, C₂H₆, C₂H₄, by employing permanently porous capsules, as depicted in FIGS. 6A and 6B, may offer significant technological advances over traditional energy intensive cryogenic distillations.

Another representative area of opportunity for compounds or tubular[n]arenes hereof is related to supercapacitors. Tubular[n]arene's redox activity, porous walls, and functional group terminus makes them good candidates in the construction of supercapacitors. As depicted in FIG. 6C, the heteroatoms forming the walls of the nanotube may serve as a locus of coordination for metals with the purpose of doping the tubular[n]arene, or as coordination sites for metals in their environment (for example, when immersed in an electrolyte solution). These redox properties, in synergy with the anchoring ability provided by the outermost rim of the nanotube, provide for the construction of modified electrodes as displayed in FIG. 6D. Covalent attachment of tubular[n]arene hereof to an electrode may, for example, be carried out by Schiff base formation using the nanotube external rim. Nanoforests produced in such a manner may be explored for supercapacitor and other applications. High capacitance is expected based on the nanoforests potential for charge accumulation of electrons and ions.

In representative studies hereof, a series of tubular[n] arene precursors were designed and synthesized as illustrated by compounds 16a, 17a, and 18a of FIG. 7A. These representative compounds have been successfully synthesized and characterized by solution methods. The aqueous solubility of such compound is relatively low. However, alternative R groups may be chosen to increase solubility. Representative types of functional groups have proved successful in improving solubility include ethylene glycol chains and trialkylsilyl substituents. With more soluble derivatives (for example, 16-18b and 16-18c) aryl-aryl coupling reactions may be used en route to formation of small tubular[n]arene members (for example, compound 2a).

FIG. 7B illustrates the synthesis and characterization of the precursor compound 16a. The compound results from the condensation of the protonated octaamino resorcinarene 4a and phenanthrene diketone 11, wherein E=C. The product was fully characterized by NMR and X-ray spectroscopy. The NMR was collected in deuterated o-DCB at a 100° C. As set forth above, to gain general synthetic information and general insight about the physical properties like solubility, the unsubstituted 9,10-phenanthrenequinone was reacted with octaamino resorcinarene 4a. The NMR spectroscopic technique showed the presence of illustrated molecule. We further confirmed synthesis of the target compound by obtaining its X-ray single crystal structure.

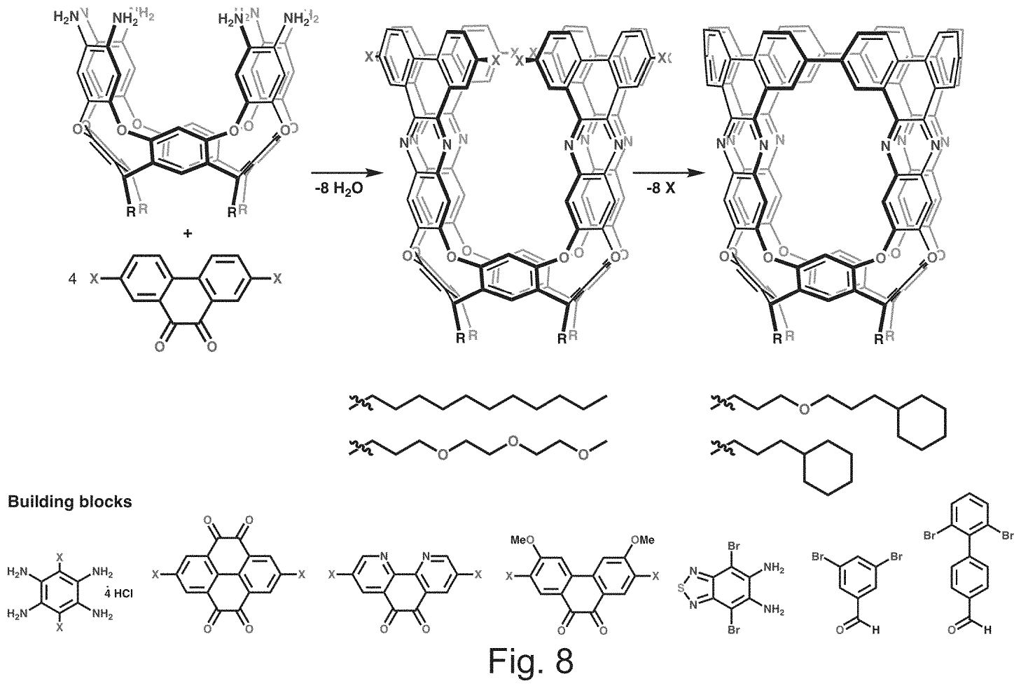

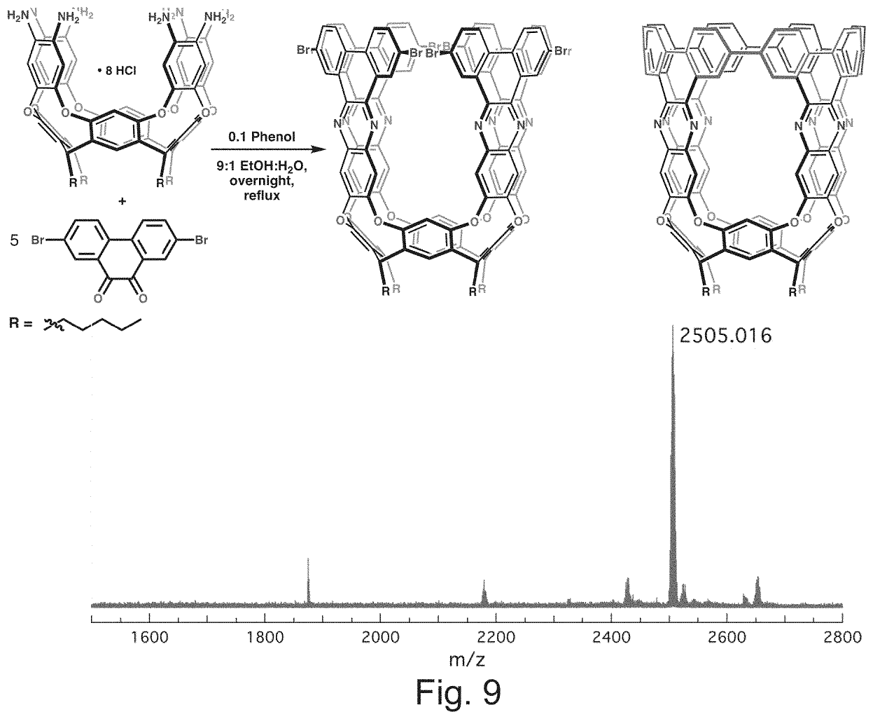

FIG. 8 illustrates another chemical representation of an embodiment of the methodology hereof. As described above, the synthetic route is based on octa-amino cavitand 4a which, for example, allows synthesis of a pyrazinic moiety or an imidazole moiety by condensation with diketo- or aldehyde-containing blocks, respectively. FIG. 8 also sets forth a number of suitable building blocks. For example, the condensation with properly halogenated (Cl, Br and I) 9,10-Phenanthrenequinone leads to the precursor which allowed a Ni-mediated cyclization reaction. The bridging alkyl unit of the end cap (resorcinarene) can be easily modified to diversify solubility. The synthesis of the final tubularene product was confirmed by MALDI-TOF-MS as illustrated in FIG. 9.

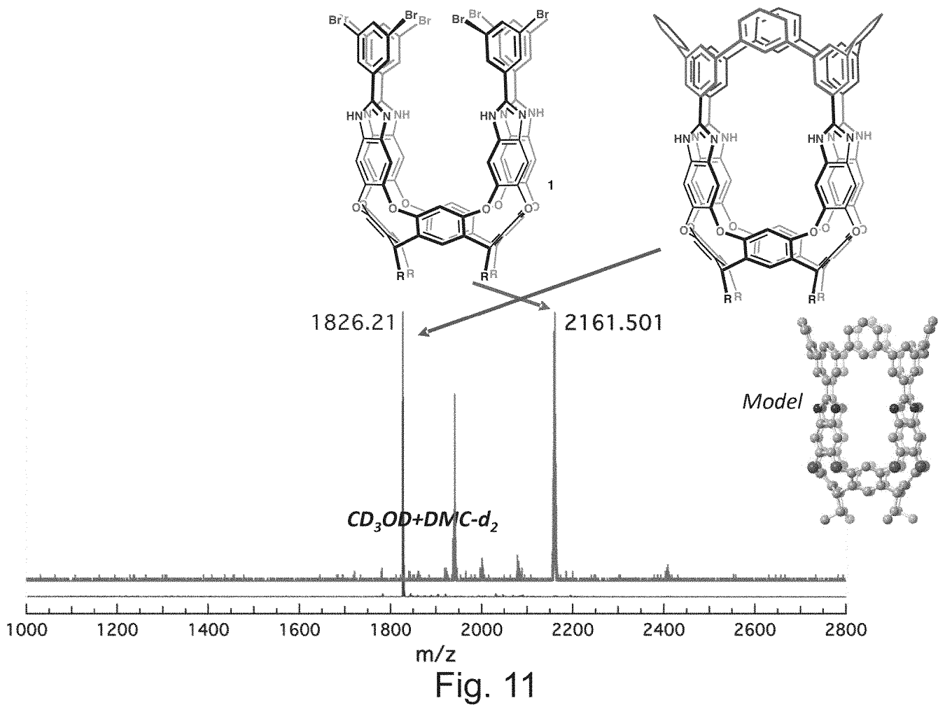

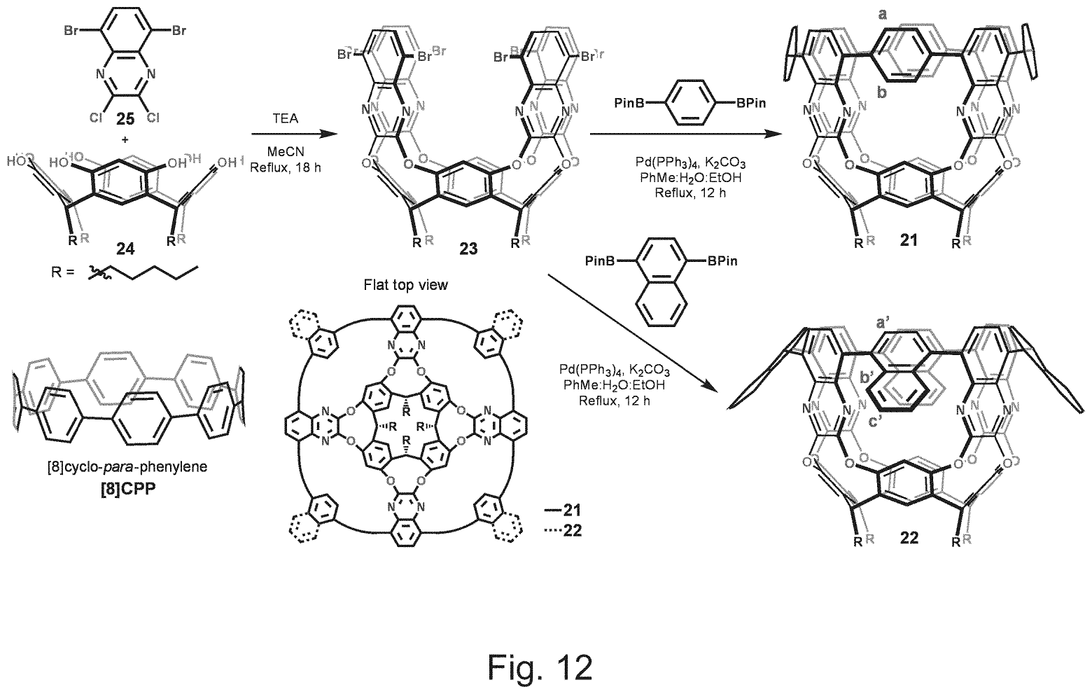

FIG. 10 illustrates another embodiment of a general route to tubular compounds hereof in which a benzaldehyde including suitable substituents as described herein is reacted in a condensation step with octa-amino cavitand 4n. In the illustrated embodiment, dibromo substitution of the benzaldehyde makes an equatorial cyclization possible. Using the dibromo benzaldehyde building block, we synthesized a new octabromo cavitand. Upon Suzuki-Miyaura cross-coupling reaction with 1,3-benzenediboronic acid bis(pinacol) ester, a zigzag top-rim cyclized tubularene was obtained. That compound has been characterized by means of MALDI-TOF (see FIG. 11) and proton NMR. It is contemplated that this synthetic approach may be used for the synthesis of cyclacene compounds. In an alternative to a Suzuki-Miyaura cross-coupling reaction with a functionalized benzene, equatorially adjacent residues of a group of building blocks hereof may, for example, be connected via an alkynyl linkage (—C≡C—) and an alkenyl linkage (—C═C—). Alkynyl linkages may also be used in axially extending the structures hereof.

FIG. 12 illustrates another embodiment of a general route to representative tubular compounds 21 and 22 hereof. As described above, these macromolecules are synthesized starting from resorcin[n]arenes as templates. Given the resulting shape and templated origin of the molecules reported herein, we termed these tubular[n_{b},n,m,r]arenes (as described above, tubularenes for short). In tubular[n_{b},n,m,r]arenes, n, (wherein b stands for basal) comes from the resorcin[nt]arene parent, n and m are the CNT chiral indices, and r corresponds to the number of aromatic rings making up the nanoring in the upper termini (disregarding the pyrazininc ring). Further description of synthesis and characterization of tubulararene (21) and tubulararene (22), as shown in FIG. 12, is set forth below.

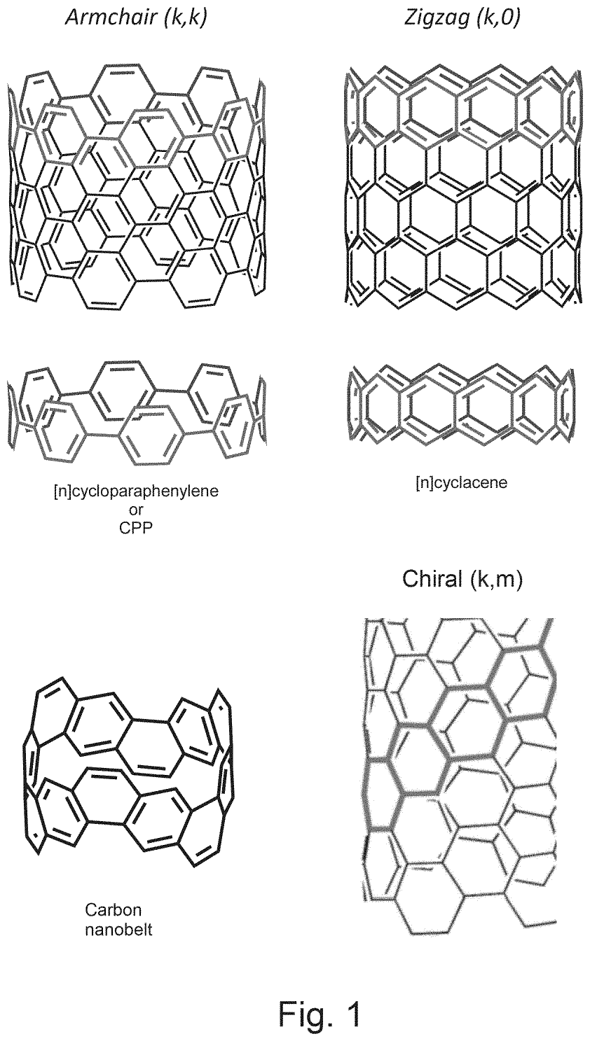

Starting materials resorcin[4]arene 24 and 2,3-dichloro-5,8-dibromoquinoxaline 25 can, for example, be synthesized on a multigram scale. Reaction between 24 and 25 under basic conditions using triethylamine (TEA) in acetonitrile under reflux produces octabromo-derivative 23 in 57% yield. Under similar reactions conditions, Suzuki-Miyaura cross coupling of 23 with either 1,4-benzenediboronic or 1,4-naphthalenediboronic acid bis(pinacol) ester affords 21 and 22, respectively. The cross-coupling cyclization step provides rigidity and aromatic conjugation to 21 and 22, much like the walls of CNTs. In 21, this newly formed upper nanoring resembles [8]CPP as described in connection with FIG. 1, although with fewer degrees of freedom since only four phenyl rings may rotate as opposed to eight in [8]CPP. Without limitation to any mechanism, it is hypothesized that this rigid conformation will prove important in maintaining electron or hole delocalization across the framework. It is known, for example, that departure from the radially oriented n-system breaks the spin density distribution in radical monocationic CPPs.

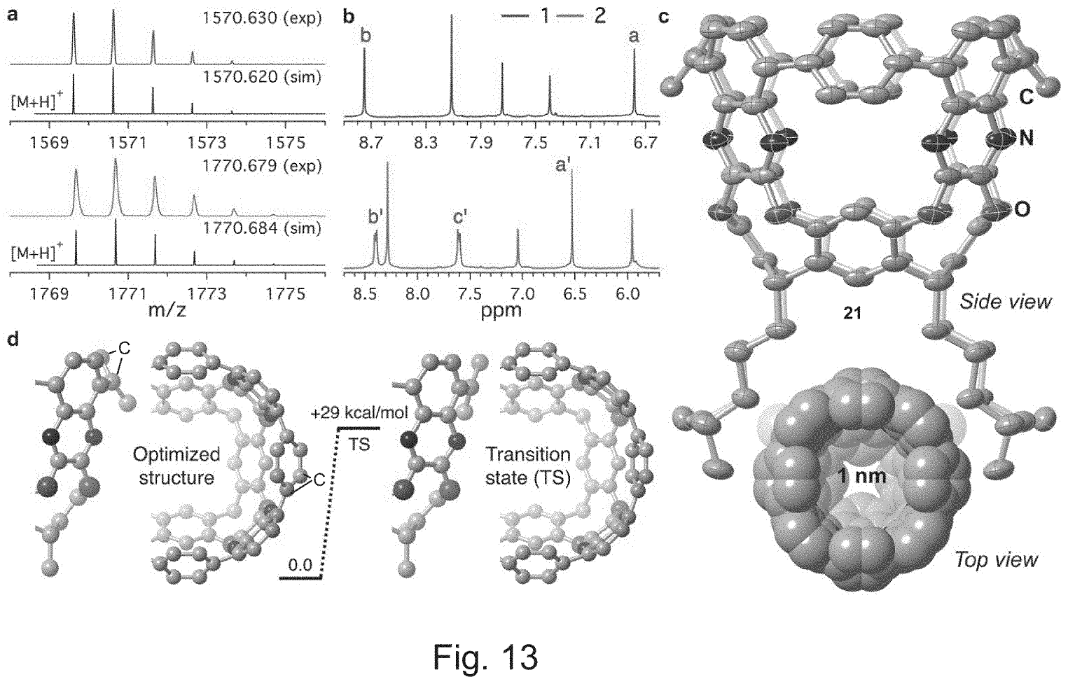

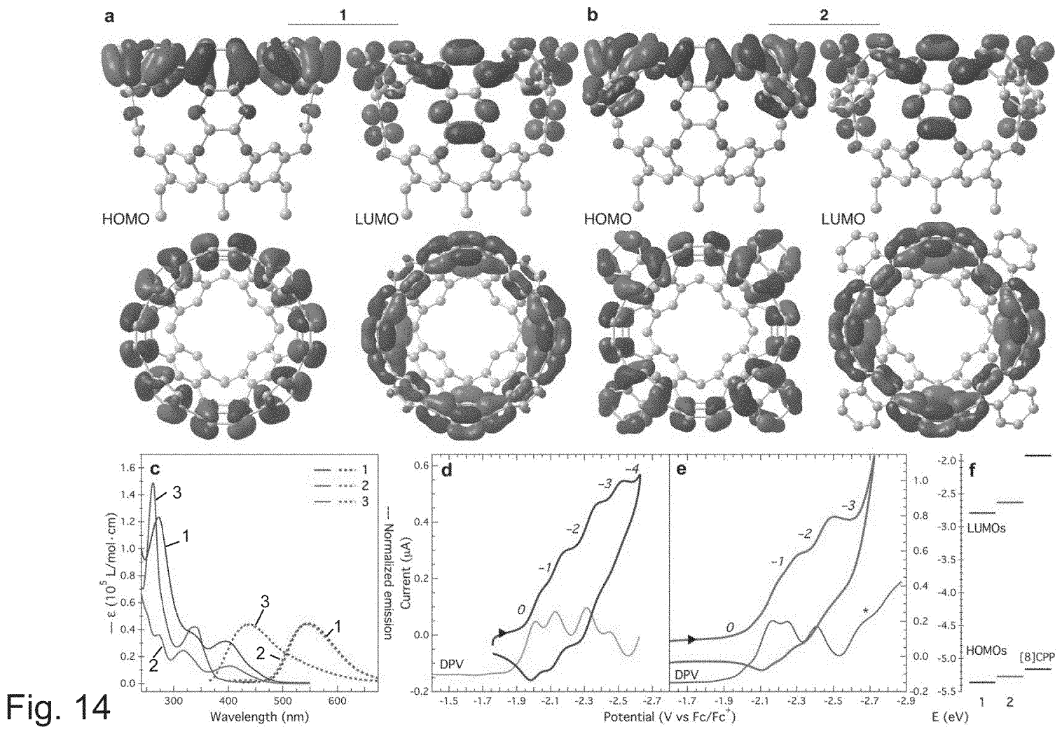

MALDI-TOF MS of tubularenes 21 and 22 display matching simulated values and isotopic distributions (FIG. 13, panel (a)), ¹H NMR of 21 and 22 is readily assigned indicating C₄ᵥ symmetry (FIG. 13, panel (b)). Slow evaporation of a dichloromethane/acetonitrile solution of 21 at room temperature provided high quality yellow-green crystals. Crystals of 22 are needle-like and weakly diffracting. The rigid framework expected for 21 was observed in its molecular crystal structure shown in FIG. 13, panel (c), and its internal cavity is readily apparent a sphere packing model. To determine the size of this cavity, we obtain a volume of ˜360 Å³, if we consider the average diameter of 21 as ˜10 Å, and having a height of ˜4.6 Å. Alternatively, by employing the solvent accessible calculator in Olex2 (crystal structure characterization software available from OlexSys Ltd of Durham UK), a volume of ˜266 Å³ is calculated.

Asset forth above, the upper nanoring of 21 resembles [8]CPP, albeit with fewer degrees of freedom. The strain energy reported for [8]CPP is 72.2 kcal/mol, obtained by DFT at the B3LYP/6-31G(d) level of theory, considering a homodesmotic reaction. Following a similar approach at the same level of theory, we determined the strain energy of 21 to be 92.4 kcal/mol. DFT calculations at different levels of theory produced consistent values around 89±3 kcal/mol. The larger value for tubularene 21 relative to [8]CPP is expected, especially since 21 has only four out of eight phenyl rings that can freely rotate. In fact, free rotation is hampered at room temperature as observed in the vastly different chemical environments of a versus b protons at 6.8 and 8.7 ppm (FIG. 13, panel (b)), respectively. DFT-calculated nucleus-independent chemical shifts (NICS) establish a shielding effect at the interior of the tubularene. We found by DFT analysis a rotational barrier of ˜29±1 kcal/mol for this phenyl group (FIG. 13, panel (d)). Comparing to other highly strained molecules, this barrier is similar in magnitude to several others where temperatures well above 150° C. are required for rotation of the aromatic group or inversion of stereochemistry.44-51 Note that in the latter examples DFT calculations reproduce closely the experimental rotational barriers. In contrast, similar phenyl rotation in [8]CPP is lower than 4 kcal/mol. Without limitation to any mechanism, it was hypothesized the locked conformation in 21 to be particularly relevant to maintaining conjugation across the tubularene. Indeed, DFT calculations show the HOMO density evenly distributed across the upper rim of 21, whereas the LUMO has significant orbital density at the pyrazinic fragments (FIG. 14, panel (a)).

With respect to optoelectronic properties, species 21 and 22 display lowest energy absorption bands at λmax of 394 (ε=31380 L/mol·cm) and 402 (ε=12970 L/mol·cm) nm, respectively. These bands are red-shifted in comparison to the lowest energy transition of precursor 23 at λmax=338 (ε=42030 L/mol·cm) nm (FIG. 14, panel (c)). Regarding emission, the fluorescence envelope of 21 and 22 is also red-shifted in comparison to precursor 23 (λem=435 nm). Emission bands of 21 and 22 are almost identical to each other only slightly shifted in λem from 546 to 542 nm, respectively. Using these data, we extracted the optical band gap (Egap) for both tubularenes. The Egap for 21 and 22 is 2.57 and 2.64 eV, respectively.

To gain further insight of the photophysical properties of compounds 21 and 22, we performed time-dependent (TD) DFT calculations at various levels of theory. For both tubularenes, we found that the HOMO-to-LUMO transition is forbidden, as in [n]CPPs. A structure-function relationship has previously been between octamethoxy-[8]CPP (λem=458 nm) and [8]CPP (?em=535 nm) indicating that a bathochromic shift in emission corresponds to increased radial π-conjugation. Extrapolating this correlation to the emissive properties of 21 and 2 supports the conclusion that the tubularene's rigidity assists in maintaining a large n-conjugated surface, although not as much as [8]CN (λem=570 nm).

Electrochemically, tubularenes 21 and 22 display a rich series of reductive events in ortho-dichlorobenzene. Cyclic voltammograms (CVs) exhibit onset of reduction at around −1.95 and −2.05 V vs Fc/Fc⁺ for 21 and 22 (FIG. 14, panels (d_ and (e)), respectively. Initially, those values were counterintuitive since as it was expected that the LUMO of 22 would be more accessible and lower in energy than 21 as a result of the larger n surface of 22. Inspection of the DFT optimized structure of 22 and its LUMO density plot shown in FIG. 14, panel (b), provides an explanation for this unexpected behavior. In that regard, as a result of the naphthylene fragments bending outwards the fragments are devoid of LUMO density, hence effectively reducing the extent of conjugation of the n surface in 22.

The CV of tubularene 21 displays four reduction events on the cathodic scan, but since these are clustered together, it is not possible to extract the half-wave potentials (E½) from the CV alone. Fortunately, by differential pulse voltammetry (DPV trace in FIG. 14, panel (d)) four peak maximums were observed at −2.01, −2.13, −2.31, and −2.45 V vs Fc/Fc⁺, each corresponding to a reduction event. In contrast to [8]CPP, oxidation events for 21 or 22 are not observed up to approximately +0.8 V vs Fc/Fc⁺. Similar to 21, tubularene 22 displays an equally crowded CV (FIG. 14, panel (e)). As in tubularene 21, DPV of 22 is well-resolved and shows three reduction peaks at −2.17, −2.25, and −2.41 V vs Fc/Fc⁺ (DPV trace in FIG. 14, panel (e)). The fourth reduction of 22 is presumed to occur concomitant with solvent reduction based on the shoulder observed at around −2.65 V vs Fc/Fc⁺ (marked with an asterisk in FIG. 14, panel (e)). However, for the same number of electrons added (q), the E½ for 22 is 100 to 150 mV more cathodic relative to 21, indicating that 22 is harder to reduce than 21.

The electrochemical LUMO levels (ELUMO) were calculated for 21 and 22 by employing E½ of the first reduction event according to ELUMO=−[E½+4.80] eV. Additionally, the HOMO energy level was obtained by subtracting Egap from ELUMO. HOMO and LUMO levels for 21 and 22 are plotted in FIG. 14, panel (f)). For comparison, the computed HOMO and LUMO positions for [8]CPP are −5.16 and −1.92 eV, respectively. Therefore, by extending the conjugation to the quinoxaline walls, while maintaining a radial orientation of the π surface, tubularene 21 and 22 are able to bring down the LUMO energy level. In contrast, HOMO-LUMO levels in [n]CPPs are determined by the number of 1,4-phenylene units making up the nanoring.

Last, to gain further insight into the rigidity effect of tubularene 21 into electron delocalization across the aromatic framework, DFT calculations were performed on the radicals formed in 21 after removing an electron from the HOMO (1+^{•}) and adding an electron to the LUMO (1^{-•}). Results show the spin density of the radical completely delocalized across the conjugated backbone of 21, in stark contrast to [8]CPP and other similar contorted aromatics, where the radical is mostly localized into a portion of the molecule.

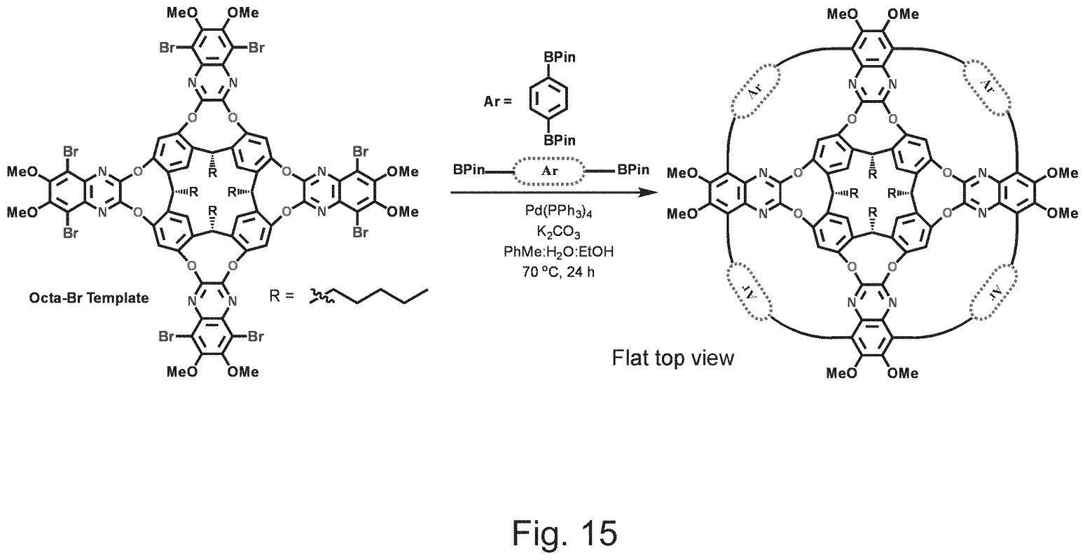

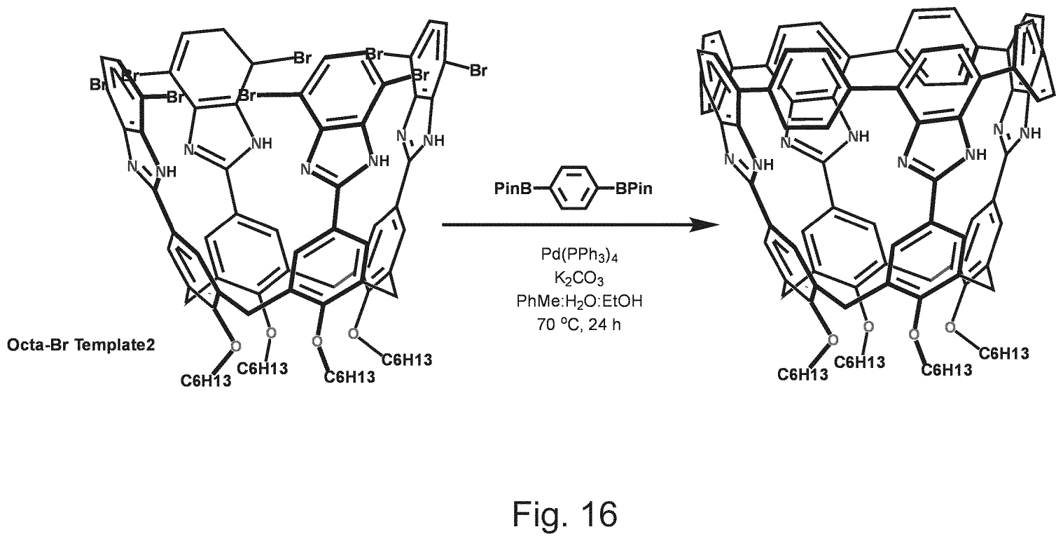

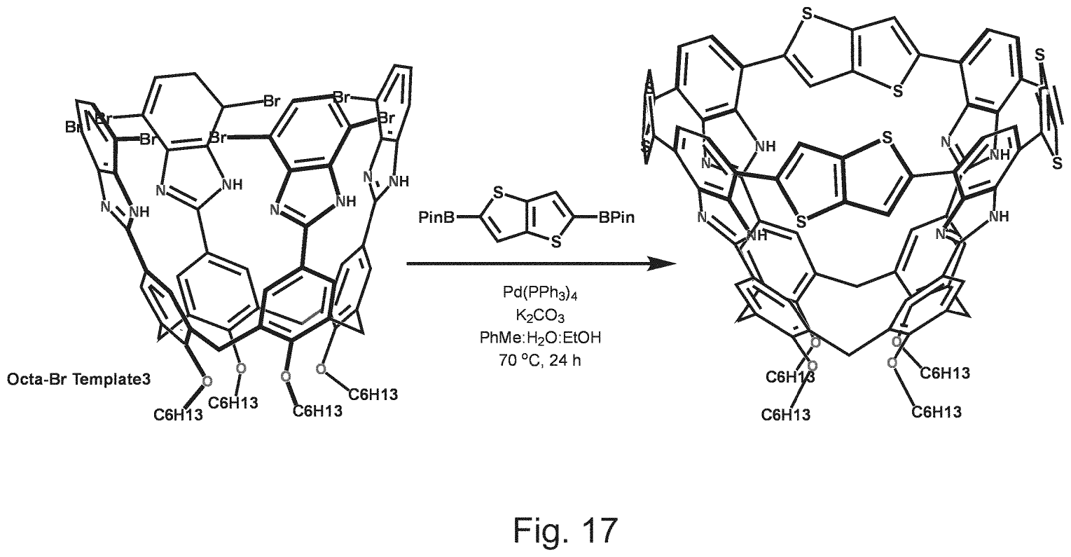

Synthesis and characterization of further tubularenes is illustrated in FIGS. 15 through 22. FIG. 15 illustrates synthesis of a tubularene from an Octa-Br end cap or template (Octa-Br template) is reacted with a cross-coupling agent (1,4-benzenediboronic acid bis(pinacol) ester). FIG. 16 illustrates synthesis of another tubularene from another Octa-Br end cap or template (Octa-Br template 2) which is reacted with the cross-coupling agent 4-benzenediboronic acid bis(pinacol) ester. FIG. 17 illustrates synthesis of a further tubularene from another Octa-Br end cap or template (Octa-Br template 2) which is reacted with the cross-coupling agent thieno[3,2-b] thiophene, 2,5-bis(4,4,5,5-tetramethyl-1,3,2-dioxaborolan-2-yl).

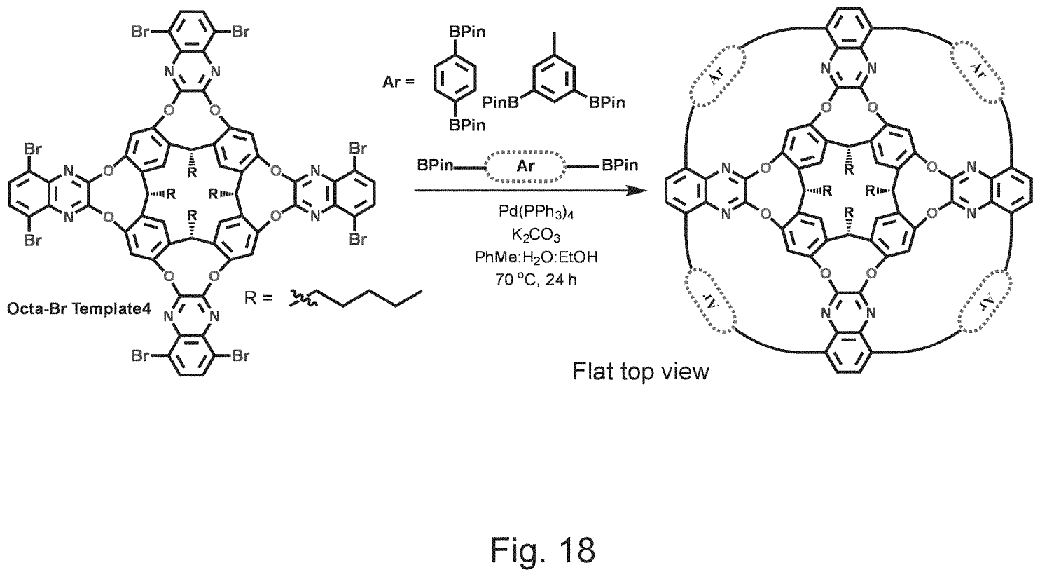

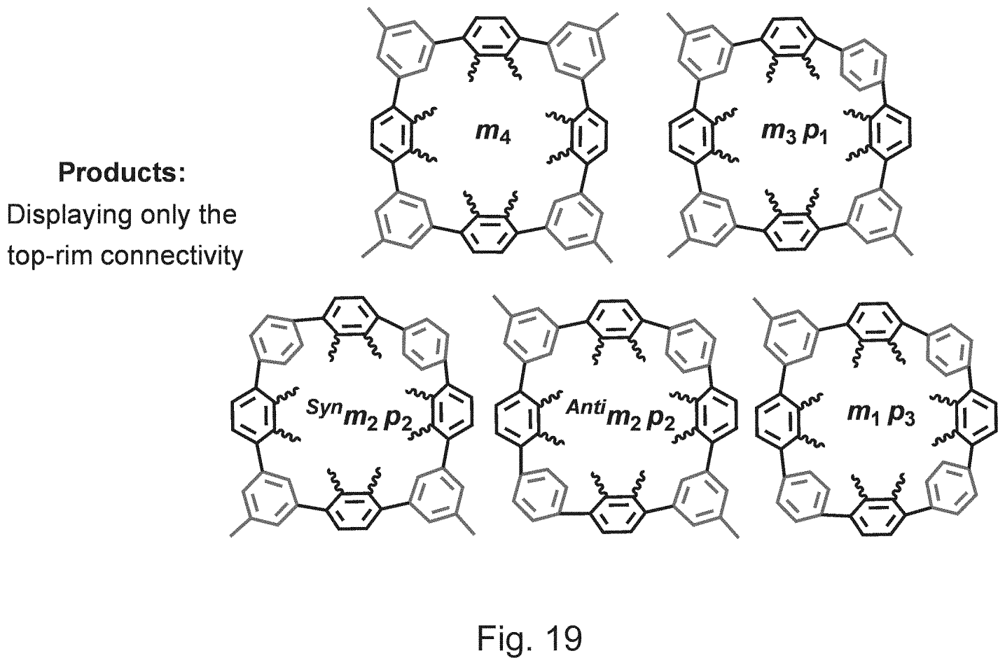

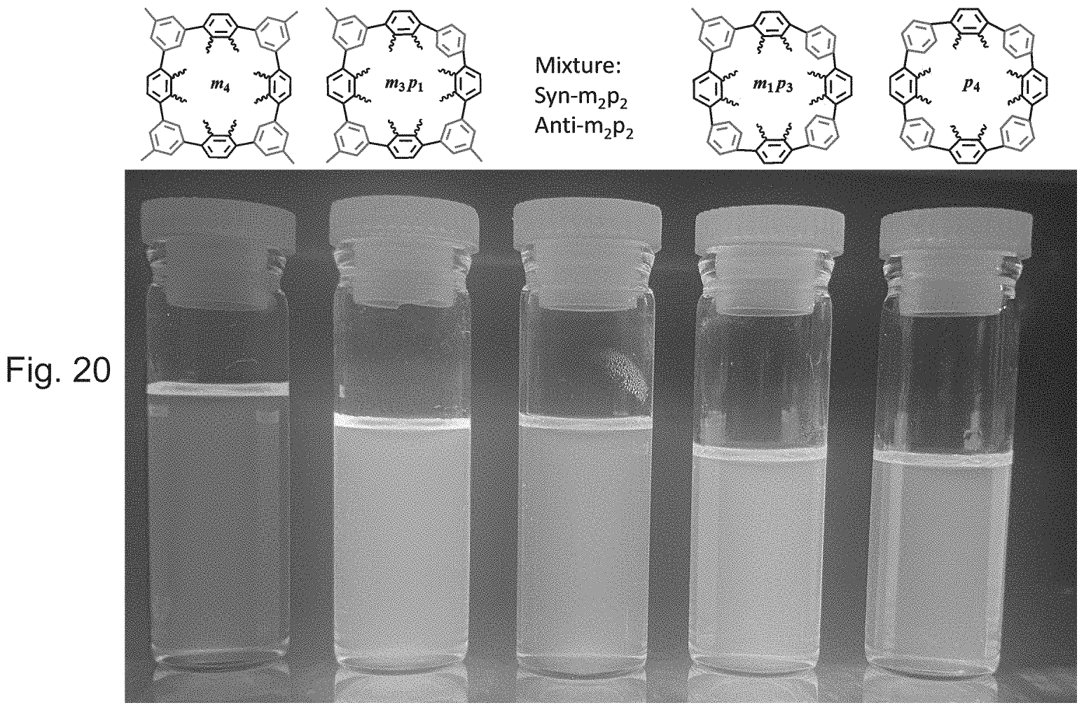

FIG. 18 illustrates synthesis of further tubular structures/compounds hereof in which another Octa-Br end cap or template (Octa-Br template 4) is reacted with the cross-coupling agents 1,4-benzenediboronic acid bis(pinacol) ester and 1,3-tolueneboronic acid bis(pinacol) ester. Top rim connectivity of the products of the reaction of FIG. 18 are illustrated in FIG. 19. FIG. 20 illustrates a shift in the fluorescence of isolated products set forth in FIG. 19. In FIG. 20 from left to right, there is a shift in the fluorescence emission of the isolated compounds from the blue to the red. The fluorescence tuning illustrated in FIG. 20 demonstrates a direct application of the methods and compositions hereof.

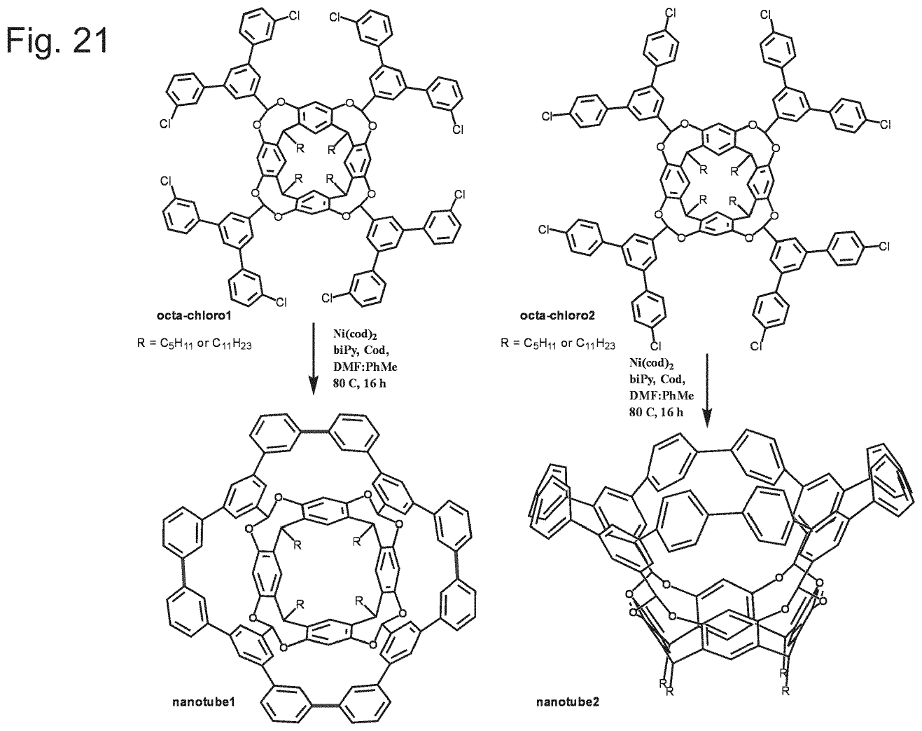

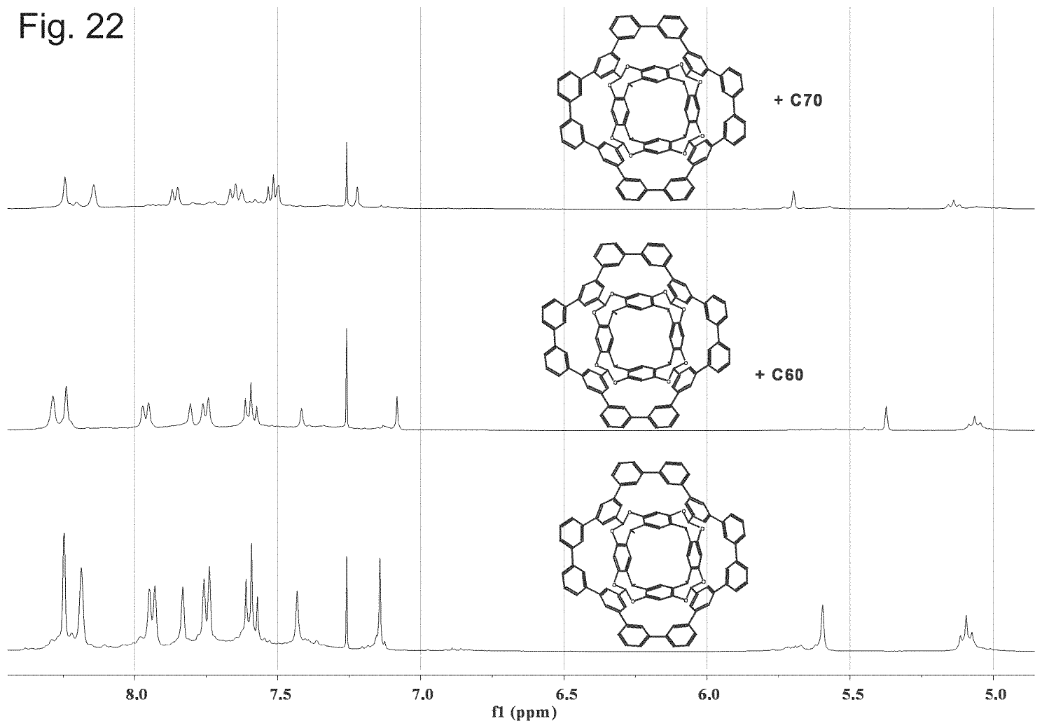

FIG. 21 illustrates synthesis of additional tubular structures/compounds hereof in which end caps or templates octa-chloro 1 and octa-chloro 2 undergo an equatorial cyclization to form compounds nanotube1 and nanotube2, respectively. FIG. 22 demonstrates that the isolated product, nanotube1, displays host-guest binding properties towards fullerenes C₆₀ and C₇₀. Such host-guest binding properties provides the potential for selective separation of the most common fullerenes. Such a result is highly desired in industry to avoid the expensive use of high-performance liquid chromatography (HPLC) in such separations.

In effecting selective separations such as separation of fullerenes, the tubular structures/compounds hereof may, for example, be reacted with substrate or structure (for example, a porous or permeable membrane) via functional R groups on the tubular structures/compound with reactive functional groups of the substrate or structure to form a separation article or structure (for example, a membrane, a surface, etc.). Alternatively, the R groups of the tubular structures/compounds hereof may be polymerizable as described above to from such a separation article or structure.

### Experimental

Synthesis of the Compound of FIG. 15.

A Pyrex Schlenk flask was loaded with 0.3 g of Octa-Br template (0.16 mmol), 0.3 g of 1,4-benzenediboronic acid bis(pinacol) ester, and 1.2 g of K₂CO₃ in 250 mL of toluene, 25 mL of water and 25 mL of EtOH. The reaction mixture was degassed for 30 min while stirring vigorously at room temperature. To this mixture, 0.18 g of Pd(PPh₃)₄ (0.16 mmol, 1 eq) was added. The solution was degassed again for an additional 15 min while gradually increasing the temperature to 70° C. This reaction was repeated three times. The solvent was removed under vacuum and the resulting solid passed through a flash column using pure DCM in hexanes. The final product was purified using 70% DCM in hexanes by preparatory TLC.

Synthesis of the Compound of FIGS. 16 and 17.

A Pyrex Schlenk flask was loaded with 0.3 g of Octa-Br template2 (0.16 mmol), 0.3 g of 1,4-benzenediboronic acid bis(pinacol) ester (or Octa-Br template3 and Thieno[3,2-b]thiophene, 2,5-bis(4,4,5,5-tetramethyl-1,3,2-dioxaborolan-2-yl) and 1.2 g of K₂CO₃ in 250 mL of toluene, 25 mL of water and 25 mL of EtOH. The reaction mixture was degassed for 30 min while stirring vigorously at room temperature. To this mixture, 0.18 g of Pd(PPh₃)₄ (0.16 mmol, I eq) was added. The solution was degassed again for an additional 15 min while gradually increasing the temperature to 70° C. This reaction was repeated three times. The solvent was removed under vacuum and the resulting solid passed through a flash column using 50% EtAc in hexanes. The final product was purified using 25% EtAc in hexanes by preparatory TLC.

Synthesis of the Compounds of FIGS. 18 and 19.

A Pyrex Schlenk flask was loaded with 0.3 g of Octa-Br template4 (0.16 mmol), 0.15 g of 1,4-benzenediboronic acid bis(pinacol) ester and 0.15 g of 1,3-toluenediboronic acid bis(pinacol)ester, and 1.2 g of K₂CO₃ in 250 mL of toluene, 25 mL of water and 25 mL of EtOH. The reaction mixture was degassed for 30 min while stirring vigorously at room temperature. To this mixture, 0.18 g of Pd(PPh₃)₄ (0.16 mmol, 1 eq) was added. The solution was degassed again for an additional 15 min while gradually increasing the temperature to 70° C. This reaction was repeated three times. The solvent was removed under vacuum and the resulting solid passed through a flash column using 70% DCM in hexanes. The final products (m4, m3p1, m2p2 and m1p3) were purified using 50% DCM in hexanes by preparatory TLC.

Synthesis of the Compounds of FIG. 21.

A mixture of 2,2′-bipyridine (21 eq), 1,5-cyclooctadiene (21 eq), and bis(1,5-cyclooctadiene)nickel(0) (21 eq) in a mixture of degassed toluene (40 mL) and DMF (40 mL) was stirred at 80° C. for 20 min. To the mixture at 80° C. was added a solution of octa-chloro compound 1 or 2 (200 mg) in toluene (20 ml) dropwise over 1 h, and the mixture was stirred at 80° C. over the night. After the reaction mixture was cooled down to ambient temperature, the solvents were dried using rotavapor and black solid passed though the flash column with pure DCM. The product has been purified using column chromatography using 50% DCM in hexanes. These reactions have been carried out with derivatives containing two different alkyl tails R═C₅H₁₁ or C₁₁H₂₃.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

What is claimed is:

1. A method of creating a tubular compound, comprising:
providing an end cap including a macrocycle that is sufficiently rigid that it presents a plurality of functional groups extending from a first axial side of the end cap;
creating a tubular wall from a plurality of reactive compounds which is covalently attached to the end cap via reaction of the plurality of functional groups on the end cap, the tubular wall extending axially from the end cap; and
performing at least one equatorial cyclization reaction axially distant from the end cap which includes covalent bonding of residues of a group of the plurality of reactive compounds used in forming the tubular wall.

2. The method of claim 1 wherein the macrocycle is generally cylindrical or ellipsoidal.

3. The method of claim 1 wherein the end cap is formed from a macrocyclic compound comprising arene groups.

4. The method of claim 3 wherein the plurality of functional groups on the first axial side of the macrocycle are hydroxyl, alkoxyl, halide or aldehyde groups.

5. The method of claim 3 wherein the macrocycle is a resorcin[n]arene wherein n is an integer between 4 and 6, a bridged resorcin[n′]arene wherein n′ is an integer between 4 and 6, or a calix[n″ ]pyrrole-resorcinarene wherein n″ is an integer between 4 and 6.

6. The method of claim 5 wherein the tubular wall includes acene groups, heteroatom-containing acene groups, alkynylene groups, or alkenylene groups.

7. The method of claim 5 wherein at least a portion of the plurality of reactive compounds include an arene group, an acene group, an N-heteroarene group or an N-heteroacene group.

8. The method of claim 3 wherein the end cap includes a macrocyclic compound including arene groups and including the plurality of functional groups extending from the first axial side thereof, the method further comprising reacting a first group of compounds of the plurality of reactive compounds with the macrocyclic compound to extend the macrocyclic compound in the axial direction, each of the first group of compounds including at least one functional group which is reactive with at least one of the plurality of functional groups of the macrocyclic compound extending from the first axial side thereof under a first set of reaction conditions, the first group of compounds of the plurality of reactive compounds further comprising at least one of (i) at least one other functional group which does not react with the plurality of functional groups of the macrocyclic compound extending from the first axial side thereof under the first set of reaction conditions and is oriented to react with one of a second group of compounds of the plurality of reactive compounds to axially extend the tubular wall; or (ii) two functional groups which do not react with the plurality of functional groups of the macrocyclic compound extending from the first axial side thereof under the first set of reaction conditions that are oriented to react to connect adjacent residues of the compounds of the first group of compounds to form the at least one equatorial cyclization.

9. The method of claim 8 wherein the compounds of the first group of compounds include the at least one functional group to react with one of the compounds of the second group of compounds and two functional groups to connect adjacent residues of the compounds of the first group of compounds to form the at least one equatorial cyclization.

10. The method of claim 9 wherein, after the compounds of the first group of compounds are reacted with the macrocyclic compound, the method further comprises reacting the second group of compounds with the residues of the first group of compounds to axially extend the tubular wall.

11. The method of claim 8 wherein the plurality of reactive compounds is selected to maintain pi conjugation axially and equatorially in the tubular wall.

12. The method of claim 8 wherein the at least one equatorial cyclization reaction is an aryl-aryl coupling, a Suzuki-Miyaura cross coupling reaction to create a linkage including a phenylene group, a reaction to create a diimide linkage, or a reaction to create an alkynyl linkage.

13. The method of claim 8 wherein a plurality of equatorial cyclization reactions is performed axially distant from the end cap.

14. The method of claim 8 wherein the macrocycle further comprises a second plurality of functional groups extending axially from a second axial side of the end cap, opposite the first side.

15. The method of claim 14 wherein the second plurality of functional groups are selected to control solubility, to participate in a polymerization reaction, or to attach the tubular compound to a substrate.

16. The method of claim 1 wherein the macrocycle is: 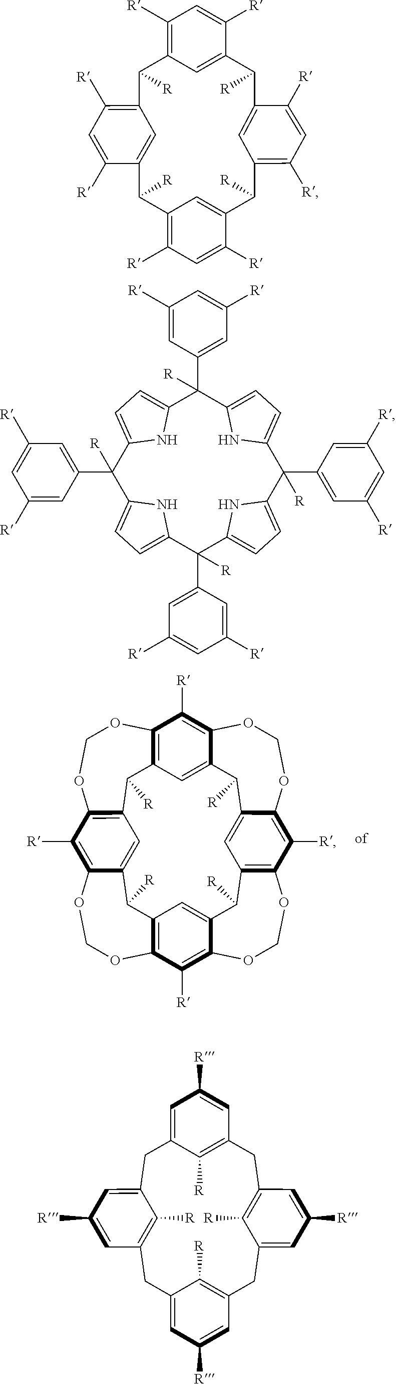
wherein R′ is a hydroxy group or an alkoxy group, R″ is a halo group or an aldehyde group, R′″ is a halo group, a hydroxy group, an amine group, an aldehyde group, or a carboxylic group, and R is an alkyl group, an aryl group, an alcohol group, an amine group, a carboxyl group, an ether group, an olefinic group, or a hydrophilic polymer.

17. A tubular compound formed by a process comprising:
providing an end cap including a macrocycle that is sufficiently rigid that it presents a plurality of functional groups extending from a first axial side of the end cap;
creating a tubular wall from a plurality of reactive compounds which is covalently attached to the end cap via reaction of the plurality of functional groups extending from the first axial side of the end cap, the tubular wall extending axially from the end cap; and
performing at least one equatorial cyclization reaction axially distant from the end cap which includes covalent bonding of residues of a group of the plurality of reactive compounds used in forming the tubular wall.

18. The tubular compound of claim 17 wherein the end cap includes a macrocyclic compound including arene groups and including the plurality of functional groups extending from a first axial side thereof, the method further comprising reacting a first group of compounds of the plurality of reactive compounds with the macrocyclic compound to extend the macrocyclic compound in the axial direction, each of the first group of compounds including at least one functional group which is reactive with at least one of the plurality of functional groups of the macrocyclic compound extending from the first axial side thereof under a first set of reaction conditions, the first group of compounds of the plurality of reactive compounds further comprising at least one of(i) at least other functional group which does not react with the plurality of functional groups of the macrocyclic compound extending from the first axial side thereof under the first set of reaction conditions and is oriented to react with one of a second group of compounds of the plurality of reactive compounds to axially extend the tubular wall; or (ii) two functional groups which do not react with the plurality of functional groups of the macrocyclic compound extending from the first axial side thereof under the first set of reaction conditions that are oriented to react to connect adjacent residues of the compounds of the first group of compounds to form the at least one equatorial cyclization.

19. The tubular compound of claim 18 wherein the compounds of the first group of compounds include the at least one functional group to react with one of the compounds of the second group of compounds and two functional groups to connect adjacent residues of the compounds of the first group of compounds to form the at least one equatorial cyclization.

20. The tubular compound of claim 19 wherein, after the compounds of the first group compounds are reacted with the macrocyclic compound, the method further comprises reacting the second group of compounds with the residues of the first group of compounds to axially extend the tubular wall.

21. The tubular compound of claim 18 wherein the plurality of reactive compounds is selected to maintain pi conjugation axially and equatorially in the tubular wall.

22. The tubular compound of claim 18 wherein the at least one equatorial cyclization reaction is an aryl-aryl coupling, a Suzuki-Miyaura cross coupling reaction to create a linkage including a phenylene group, a reaction to create a diimide linkage, or a reaction to create an alkynyl linkage.

23. The tubular compound of claim 18 wherein a plurality of equatorial cyclization reactions is performed axially distant from the end cap.

24. The tubular compound of claim 18 wherein the macrocycle further comprises a second plurality of functional groups extending from a second axial side of the end cap, opposite the first side.

25. The tubular compound of claim 24 wherein the second plurality of functional groups are selected to control solubility, to participate in a polymerization reaction, or to attach the tubular compound to a substrate.

26. The tubular compound of claim 18 wherein the macrocycle is: 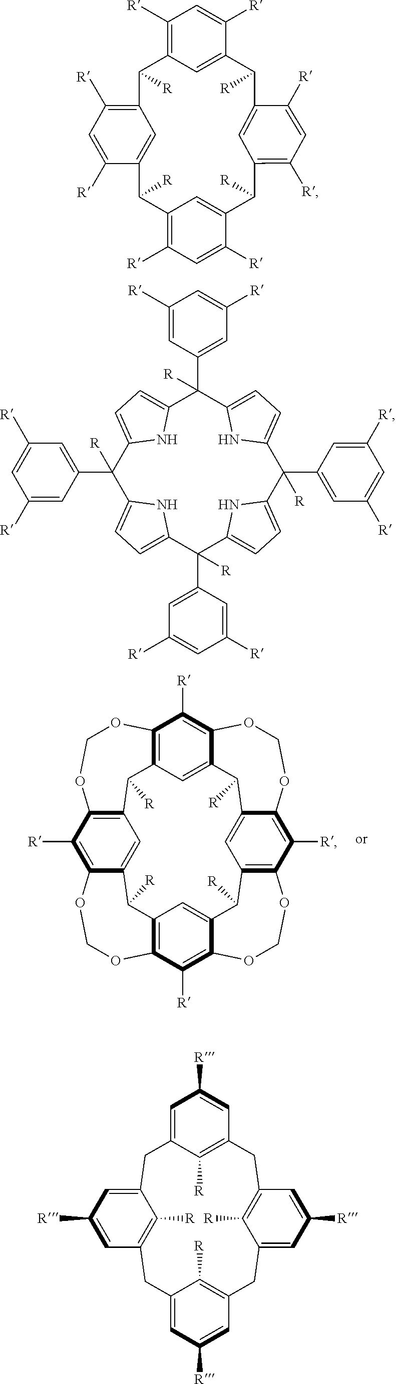
wherein R′ is a hydroxy group or an alkoxy group, R″ is a halo group or an aldehyde group, R′″ is a halo group, a hydroxy group, an amine group, an aldehyde group, or a carboxylic group, and R is an alkyl group, an aryl group, an alcohol group, an amine group, a carboxyl group, an ether group, an olefinic group, or a hydrophilic polymer.

27. The tubular compound of claim 26 having the formula

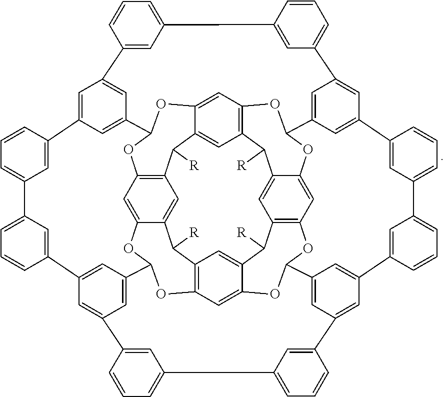

28. A structure for use in a separation, comprising tubular residues of a tubular compound formed by a process comprising:
providing an end cap including a macrocycle that is sufficiently rigid that it presents a plurality of functional groups extending from a first axial side of the end cap, the macrocycle further including a plurality of functional groups extending from a second axial side of the end cap, opposite the first axial side, wherein reaction of one or more of the plurality of functional groups extending from the second axial side of the end cap covalently bonds the tubular residues of the tubular compounds within the structure;
creating a tubular wall from a plurality of reactive compounds which is covalently attached to the end cap via reaction of the plurality of functional groups extending from the first axial side of the end cap, the tubular wall extending axially from the end cap;
performing at least one equatorial cyclization reaction axially distant from the end cap which includes covalent bonding of residues of a group of the plurality of reactive compounds used in forming the tubular wall; and
reacting one or more of the plurality of functional group extending from the second axial side of the end cap to covalently bond the tubular residues of the tubular compounds within the structure.

29. The structure of claim 28 wherein one or more of the plurality of functional group extending from the second axial side of the end cap are reacted with one or more functional groups of a substrate for the structure in forming the structure, or one or more of the plurality of functional group oriented to extend from the second axial side of the end cap are reacted in a polymerization reaction in forming the structure.

30. The structure of claim 28 wherein the end cap includes a macrocyclic compound including arene groups and including the plurality of functional groups extending from the first axial side thereof, and wherein the tubular compound are formed by reacting a first group of compounds of the plurality of reactive compounds with the macrocyclic compound to extend the macrocyclic compound axially from the first axial side thereof, each of the first group of compounds including at least one functional group which is reactive with at least one of the plurality of functional groups of the macrocyclic compound extending from the first axial side thereof under a first set of reaction conditions, the first group of compounds of the plurality of reactive compounds further comprising at least one of (i) at least other functional group which does not react with the plurality of functional groups of the macrocyclic compound extending from the first axial side thereof under the first set of reaction conditions and is oriented to react with one of a second group of compounds of the plurality of reactive compounds to axially extend the tubular wall; or (ii) two functional groups which do not react with the plurality of functional groups of the macrocyclic compound extending from the first axial side thereof under the first set of reaction conditions that are oriented to react to connect adjacent residues of the compounds of the first group of compounds to form the at least one equatorial cyclization.

31. The structure of claim 30 wherein the macrocycle is: 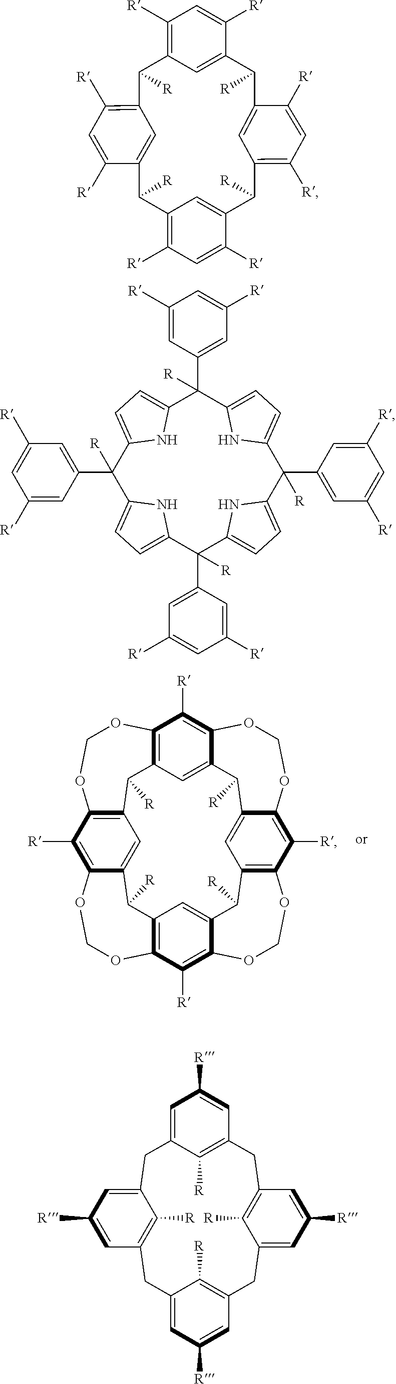
wherein R′ is a hydroxy group or an alkoxy group, R″ is a halo group or an aldehyde group, R′″ is a halo group, a hydroxy group, an amine group, an aldehyde group, or a carboxylic group, and R is an alkyl group, an aryl group, an alcohol group, an amine group, a carboxyl group, an ether group, an olefinic group, or a hydrophilic polymer.

32. The structure of claim 31 wherein the tubular residue of the tubular compound selectively interacts with at least one compound to be separated from a mixture of compounds.

33. The structure of claim 31 wherein the tubular residue of the tubular compound selectively interacts with at least one fullerene to be separated from a mixture of different fullerenes.

34. The structure of claim 32 wherein the tubular compound has the formula [chemical structure]
